(19) United States
(12) Patent Application Publication
(10) Pub. No.: US 2023/0347172 A1
(43) Pub. Date: Nov. 2, 2023

(54) RADIONUCLIDE BRACHYTHERAPY SOURCE SYSTEMS FOR APPLICATION OF BETA RADIATION

(71) Applicant: RADIANCE THERAPEUTICS, INC., Tucson, AZ (US)

(72) Inventors: Laurence J. Marsteller, Tucson, AZ (US); David Gyorke, Tucson, AZ (US); Alan Chamberlain, Tucson, AZ (US); Wendell Lutz, Tucson, AZ (US)

(21) Appl. No.: 18/337,367

(22) Filed: Jun. 19, 2023

Related U.S. Application Data
(63) Continuation-in-part of application No. PCT/US2021/064141, filed on Dec. 17, 2021.
Application No. PCT/US2021/064141 is a continuation-in-part of application No. PCT/US2021/012694, filed on Jan. 8, 2021.
Application No. PCT/US2021/012694 is a continuation-in-part of application No. PCT/US2021/012744, filed on Jan. 8, 2021.
Application No. PCT/US2021/012744 is a continuation-in-part of application No. 17/861,096, filed on Jul. 8, 2022.
Application No. 17/861,096 is a continuation-in-part of application No. PCT/US2021/012744, filed on Jan. 8, 2021.
Application No. PCT/US2021/012744 is a continuation-in-part of application No. 17/861,069, filed on Jul. 8, 2022.
Application No. 17/861,069 is a continuation-in-part of application No. PCT/US2021/012694, filed on Jan. 8, 2021.
Application No. 17/861,069 is a continuation-in-part of application No. PCT/US2021/012744, filed on Jan. 8, 2021.
Application No. 17/861,096 is a continuation-in-part of application No. PCT/US2021/012694, filed on Jan. 8, 2021.
Application No. PCT/US2021/012694 is a continuation-in-part of application No. 17/694,366, filed on Mar. 14, 2022.
Application No. 17/694,366 is a continuation-in-part of application No. 16/698,676, filed on Nov. 27, 2019, now Pat. No. 11,273,325.
Application No. 17/694,366 is a continuation-in-part of application No. PCT/US2021/012744, filed on Jan. 8, 2021.
Application No. PCT/US2021/012744 is a continuation-in-part of application No. PCT/US2021/012694, filed on Jan. 8, 2021.
Application No. 17/861,069 is a continuation-in-part of application No. 17/694,366, filed on Mar. 14, 2022.
Application No. 17/861,096 is a continuation-in-part of application No. 17/694,366, filed on Mar. 14, 2022.
Application No. 17/694,366 is a continuation-in-part of application No. 17/676,711, filed on Feb. 21, 2022.
Application No. 17/676,711 is a continuation-in-part of application No. PCT/US2020/047235, filed on Aug. 20, 2020.
Application No. 17/676,711 is a continuation-in-part of application No. PCT/US2021/012744, filed on Jan. 8, 2021.
Application No. PCT/US2021/012744 is a continuation-in-part of application No. PCT/US2021/012694, filed on Jan. 8, 2021.
Application No. PCT/US2021/012694 is a continuation-in-part of application No. 16/698,676, filed on Nov. 27, 2019, now Pat. No. 11,273,325.
Application No. 17/694,366 is a continuation-in-part of application No. 17/676,711, filed on Feb. 21, 2022.
Application No. 17/861,069 is a continuation-in-part of application No. 17/676,711, filed on Feb. 21, 2022.
Application No. 17/861,096 is a continuation-in-part of application No. 17/676,711, filed on Feb. 21, 2022.
Application No. 17/861,069 is a continuation-in-part of application No. PCT/US2021/064141, filed on Dec. 17, 2021.
Application No. 17/861,096 is a continuation-in-part of application No. PCT/US2021/064141, filed on Dec. 17, 2021.
Application No. 17/694,366 is a continuation-in-part of application No. PCT/US2021/064141, filed on Dec. 17, 2021.
Application No. 17/676,711 is a continuation-in-part of application No. PCT/US2021/064141, filed on Dec. 17, 2021.
Application No. 17/861,069 is a continuation-in-part of application No. PCT/US2021/064190, filed on Dec. 17, 2021.
Application No. PCT/US2021/064190 is a continuation-in-part of application No. PCT/US2021/012744, filed on Jan. 8, 2021.
Application No. PCT/US2021/012744 is a continuation-in-part of application No. PCT/US2021/012694, filed on Jan. 8, 2021.
Application No. 17/861,096 is a continuation-in-part of application No. PCT/US2021/064190, filed on Dec. 17, 2021.
Application No. 17/694,366 is a continuation-in-part of application No. PCT/US2021/064190, filed on Dec. 17, 2021.
Application No. 17/676,711 is a continuation-in-part of application No. PCT/US2021/064190, filed on Dec. 17, 2021.
Application No. PCT/US2021/064190 is a continuation-in-part of application No. 17/782,940, filed on Jun. 6, 2022.
Application No. 17/694,366 is a continuation-in-part of application No. PCT/US2020/063435, filed on Dec. 4, 2020.
Application No. 17/676,711 is a continuation-in-part of application No. PCT/US2020/063435, filed on Dec. 4, 2020.
Application No. 17/861,069 is a continuation-in-part of application No. 17/782,940, filed on Jun. 6, 2022.
Application No. 17/861,096 is a continuation-in-part of application No. 17/782,940, filed on Jun. 6, 2022.
Application No. 17/782,940 is a continuation-in-part of application No. 18/296,825, filed on Apr. 6, 2023.
Application No. 18/296,825 is a continuation of application No. 16/584,737, filed on Sep. 26, 2019, now Pat. No. 11,666,780.
Application No. 16/584,737 is a continuation-in-part of application No. PCT/US2018/049400, filed on Sep. 4, 2018.
Application No. 17/694,366 is a continuation-in-part of application No. 16/584,737, filed on Sep. 26, 2019, now Pat. No. 11,666,780.
Application No. 17/676,711 is a continuation-in-part of application No. 16/584,737, filed on Sep. 26, 2019, now Pat. No. 11,666,780.
Application No. 17/861,069 is a continuation-in-part of application No. 16/584,737, filed on Sep. 26, 2019, now Pat. No. 11,666,780.
Application No. 17/861,096 is a continuation-in-part of application No. 16/584,737, filed on Sep. 26, 2019, now Pat. No. 11,666,780.
Application No. 16/584,737 is a continuation-in-part of application No. 18/295,074, filed on Apr. 3, 2023.
Application No. 18/295,074 is a continuation of application No. 16/810,204, filed on Mar. 5, 2020, now Pat. No. 11,628,310.
Application No. 16/810,204 is a continuation-in-part of application No. PCT/US2018/049400, filed on Sep. 4, 2018.
Application No. 17/694,366 is a continuation-in-part of application No. 16/810,204, filed on Mar. 5, 2020, now Pat. No. 11,628,310.
Application No. 17/676,711 is a continuation-in-part of application No. 16/810,204, filed on Mar. 5, 2020, now Pat. No. 11,628,310.
Application No. 17/861,069 is a continuation-in-part of application No. 16/810,204, filed on Mar. 5, 2020, now Pat. No. 11,628,310.
Application No. 17/861,096 is a continuation-in-part of application No. 16/810,204, filed on Mar. 5, 2020, now Pat. No. 11,628,310.
(60) Provisional application No. 63/126,855, filed on Dec. 17, 2020.
(60) Provisional application No. 63/486,568, filed on Feb. 23, 2023.
(60) Provisional application No. 62/958,554, filed on Jan. 8, 2020.
(60) Provisional application No. 62/958,517, filed on Jan. 8, 2020.
(60) Provisional application No. 62/958,634, filed on Jan. 8, 2020.
(60) Provisional application No. 62/772,741, filed on Nov. 29, 2018.
(60) Provisional application No. 62/889,461, filed on Aug. 20, 2019.
(60) Provisional application No. 63/126,855, filed on Dec. 17, 2020.
(60) Provisional application No. 62/944,952, filed on Dec. 6, 2019.
(60) Provisional application No. 62/738,573, filed on Sep. 28, 2018.

(30) Foreign Application Priority Data
Sep. 7, 2017 (GB) 1714392.6

(51) Int. Cl. A61N 5/10; A61M 1/00

(52) U.S. Cl. CPC A61N 5/1017; A61M 1/84; A61M 2210/0612

(57) ABSTRACT

Radionuclide brachytherapy sources and systems for applying beta radiation to a target area, for example for maintaining functioning drainage blebs or functioning drainage holes in the eye, e.g., to reduce intraocular pressure (IOP) of an eye being treated for glaucoma. The systems herein provide a substantially uniform dose across a particular area, thereby providing appropriate radiation therapies.

### CROSS-REFERENCES TO RELATED APPLICATIONS

This application is a continuation-in-part of and claims priority to PCT Application No. PCT/US2021/064141 filed Dec. 17, 2021, which claims priority to PCT Application No. PCT/US2021/012694 filed Jan. 8, 2021 and PCT Application No. PCT/US2021/012744 filed Jan. 8, 2021, the specification(s) of which is/are incorporated herein in their entirety by reference. PCT/US2021/064141 also claims priority to U.S. Provisional Application No. 63/126,855 filed Dec. 17, 2020, the specification(s) of which is/are incorporated herein in their entirety by reference.

This application is a non-provisional of and claims priority to U.S. Provisional Application No. 63/486,568 filed Feb. 23, 2023, the specifications of which are incorporated herein in their entirety by reference.

This application is a continuation-in-part and claims priority to U.S. patent application Ser. No. 17/861,096 filed Jul. 8, 2022, the specification(s) of which is/are incorporated herein in their entirety by reference. U.S. Ser. No. 17/861,096 is a continuation-in-part of and claims priority to PCT Application No. PCT/US2021/012744 filed on Jan. 8, 2021, which claims priority to U.S. Provisional Application No. 62/958,554 filed on Jan. 8, 2020, the specification(s) of which is/are incorporated herein in their entirety by reference.

This application is a continuation-in-part of and claims priority to U.S. patent application Ser. No. 17/861,069 filed Jul. 8, 2022, the specification(s) of which is/are incorporated herein in their entirety by reference. U.S. Ser. No. 17/861,069 is a continuation-in-part of and claims priority to PCT Application No. PCT/US2021/012694 filed on Jan. 8, 2021, which claims priority to U.S. Provisional Application No. 62/958,517 filed Jan. 8, 2020 and U.S. Provisional Application No. 62/958,634 filed Jan. 8, 2020, the specification(s) of which is/are incorporated herein in their entirety by reference. U.S. Ser. No. 17/861,069 is a continuation-in-part of and claims priority to PCT/US2021/012744. U.S. Ser. No. 17/861,096 is a continuation-in-part of and claims priority to PCT/US2021/012694.

This application is a continuation-in-part of and claims priority to U.S. patent application Ser. No. 17/694,366 filed on Mar. 14, 2022, the specification(s) of which is/are incorporated herein in their entirety by reference. U.S. Ser. No. 17/694,366 is a continuation-in-part of and claims priority to U.S. patent application Ser. No. 16/698,676 filed on Nov. 27, 2019, now U.S. Pat. No. 11,273,325, which claims priority to U.S. Provisional Application No. 62/772,741 filed on Nov. 29, 2018, the specification(s) of which is/are incorporated herein in their entirety by reference.

U.S. Ser. No. 17/694,366 is also a continuation-in-part of and claims priority to PCT/US2021/012744. U.S. Ser. No. 17/694,366 is also a continuation-in-part of and claims priority to PCT/US2021/012694. U.S. Ser. No. 17/861,069 is also a continuation-in-part of and claims priority to U.S. Ser. No. 17/694,366. U.S. Ser. No. 17/861,096 is also a continuation-in-part of and claims priority to U.S. Ser. No. 17/694,366.

This application is a continuation-in-part and claims priority to U.S. patent application Ser. No. 17/676,711 filed on Feb. 21, 2022, the specification(s) of which is/are incorporated herein in their entirety by reference. U.S. Ser. No. 17/676,711 is a continuation-in-part of and claims priority to PCT Application No. PCT/US2020/047235 filed on Aug. 20, 2020, which claims priority to U.S. Provisional Application No. 62/889,461 filed on Aug. 20, 2019, the specification(s) of which is/are incorporated herein in their entirety by reference.

U.S. Ser. No. 17/676,711 is also a continuation-in-part of and claims priority to PCT/US2021/012744. U.S. Ser. No. 17/676,711 is also a continuation-in-part of and claims priority to PCT/US2021/012694. U.S. Ser. No. 17/676,711 is also a continuation-in-part of and claims priority to U.S. Ser. No. 16/698,676. U.S. Ser. No. 17/694,366 is also a continuation-in-part of and claims priority to U.S. Ser. No. 17/676,711. U.S. Ser. No. 17/861,069 is also a continuation-in-part of and claims priority to U.S. Ser. No. 17/676,711. U.S. Ser. No. 17/861,096 is also a continuation-in-part of and claims priority to U.S. Ser. No. 17/676,711.

U.S. Ser. No. 17/861,069 is also a continuation-in-part of and claims priority to PCT Application No. PCT/US2021/064141 filed on Dec. 17, 2021, which also claims priority to PCT/US2021/012744, PCT/US2021/012694, and U.S. Provisional Application No. 63/126,855 filed on Dec. 17, 2020, the specification(s) of which is/are incorporated herein in their entirety by reference.

U.S. Ser. No. 17/861,096 is also a continuation-in-part of and claims priority to PCT/US2021/064141. U.S. Ser. No. 17/694,366 is also a continuation-in-part of and claims priority to PCT/US2021/064141. U.S. Ser. No. 17/676,711 is also a continuation-in-part of and claims priority to PCT/US2021/064141.

U.S. Ser. No. 17/861,069 is also a continuation-in-part of and claims priority to Application No. PCT/US2021/064190 filed on Dec. 17, 2021, which also claims priority to PCT/US2021/012744, PCT/US2021/012694, and U.S. Provisional Application No. 63/126,855.

U.S. Ser. No. 17/861,096 is also a continuation-in-part of and claims priority to PCT/US2021/064190. U.S. Ser. No. 17/694,366 is also a continuation-in-part of and claims priority to PCT/US2021/064190. U.S. Ser. No. 17/676,711 is also a continuation-in-part of and claims priority to PCT/US2021/064190.

This application is a continuation-in-part of and claims priority to U.S. Ser. No. 17/782,940 filed Jun. 6, 2022, which is a 371 application of PCT Application No. PCT/US2020/063435 filed on Dec. 4, 2020, which claims priority to U.S. Provisional Application No. 62/944,952 filed on Dec. 6, 2019, the specification(s) of which is/are incorporated herein in their entirety by reference. U.S. Ser. No. 17/694,366 is also a continuation-in-part of and claims priority to PCT/US2020/063435.

U.S. Ser. No. 17/676,711 is also a continuation-in-part of and claims priority to PCT/US2020/063435. U.S. Ser. No. 17/861,069 is also a continuation-in-part of and claims priority to U.S. Ser. No. 17/782,940. U.S. Ser. No. 17/861,096 is also a continuation-in-part of and claims priority to U.S. Ser. No. 17/782,940.

This application is a continuation-in-part of and claims priority to U.S. patent application Ser. No. 18/296,825 filed Apr. 6, 2023, which is a continuation of and claims priority to U.S. patent application Ser. No. 16/584,737 filed Sep. 26, 2019, now U.S. Pat. No. 11,666,780, which is a non-provisional and claims priority to U.S. Provisional Application No. 62/738,573 filed Sep. 28, 2018, the specifications of which are incorporated herein in their entirety by reference. U.S. Ser. No. 16/584,737 is also a continuation-in-part and claims priority to PCT Application No. PCT/US2018/049400 filed on Sep. 4, 2018, which claims priority to GB Application No. 1714392.6 filed Sep. 7, 2017, the specification(s) of which is/are incorporated herein in their entirety by reference.

U.S. Ser. No. 17/694,366 is also a continuation-in-part of and claims priority to U.S. Ser. No. 16/584,737. U.S. Ser. No. 17/676,711 is also a continuation-in-part of and claims priority to U.S. Ser. No. 16/584,737. U.S. Ser. No. 17/861,069 is also a continuation-in-part of and claims priority to U.S. Ser. No. 16/584,737. U.S. Ser. No. 17/861,096 is also a continuation-in-part of and claims priority to U.S. Ser. No. 16/584,737.

This application is a continuation-in-part of and claims priority to U.S. patent application Ser. No. 18/295,074 filed Apr. 3, 2023, which is a continuation of and claims priority to U.S. patent application Ser. No. 16/810,204, filed Mar. 5, 2020, now U.S. Pat. No. 11,628,310, which is a continuation-in-part of and claims priority to PCT Application No. PCT/US2018/049400 filed Sep. 4, 2018, which claims priority to GB Patent Application No. 1714392.6 filed Sep. 7, 2017, the specifications of which are incorporated herein in their entirety by reference.

U.S. Ser. No. 17/694,366 is also a continuation-in-part of and claims priority to U.S. Ser. No. 16/810,204. U.S. Ser. No. 17/676,711 is also a continuation-in-part of and claims priority to U.S. Ser. No. 16/810,204. U.S. Ser. No. 17/861,069 is also a continuation-in-part of and claims priority to U.S. Ser. No. 16/810,204. U.S. Ser. No. 17/861,096 is also a continuation-in-part of and claims priority to U.S. Ser. No. 16/810,204.

### BACKGROUND OF THE INVENTION

### Field of the Invention

The present invention relates to radionuclide brachytherapy sources (RBSs), RBS systems and devices, and methods for applying beta radiation to a target. The RBSs, RBS systems, and methods described herein may be used for treating glaucoma treatment-associated drainage blebs, such as those associated with foreign bodies or other glaucoma procedures, so as to maintain functioning blebs. The present invention is not limited to these applications.

### Background Art

### Glaucoma

Glaucoma is the leading cause of irreversible blindness and represents a family of diseases with a characteristic optic neuropathy. Therapy for this group of diseases is principally focused at reducing the intraocular pressure (IOP) of the fluid inside the eye (aqueous humor), thus averting ongoing damage to the optic nerve.

Glaucoma is managed by attempting to lower the intraocular pressure (IOP). In the USA, Europe, and some other industrialized countries, the first line therapy is typically medication delivered by eye drops. Such medications include beta-blockers, prostaglandins, alpha-adrenergic agonists, and carbonic anhydrase inhibitors. For patients who fail medication and in other parts of the world where there are economic and distribution barriers to the practicality of daily medication and frequent follow up, the treatment regime is primarily surgical interventions.

One way to prevent vision loss from glaucoma is to lower intraocular pressure with drainage surgery that shunts fluid out of the eye through a channel created during a trabeculectomy procedure, by implanting a flow-controlled drainage device during Minimally Invasive Glaucoma Surgery (MIGS), or by the use of other surgical procedures such as Minimally Invasive Micro Sclerostomy (MIMS), trabeculectomy, or other devices. These systems and procedures allow drainage of the aqueous humor from within the eye to a small reservoir (termed a “bleb”) under the conjunctiva, from where the aqueous humor is later reabsorbed.

With current glaucoma treatments (e.g., MIMS, MIGS, trabeculectomy, etc.), scar tissue often compromises the bleb or other surrounding structures (e.g., drainage channels associated with MIMS), ultimately impeding or blocking the flow of excess fluid. Despite compelling therapeutic advantages over nonsurgical treatments, drainage surgery and devices are clinically limited by postoperative scarring.

Attempts to address this include the application of antimetabolites such as mitomycin C (MMC) and 5-fluorouracil (5FU). These antimetabolites are used in liquid form and are delivered either by injection or by placing microsurgical sponges soaked in the drug directly onto the operative site underneath the conjunctiva. One of the problems associated with antimetabolites (e.g., MMC and 5FU) is that they do not preserve blebs well. According to some reports, the failure rate by three years approaches 50%.

### Beta Ophthalmic Applicators

Brachytherapy involves the placement of a radioisotope inside or next to the area requiring treatment, and has shown safety and efficacy in the clinical management of many diseases. Recently beta brachytherapy has been surprisingly found to be an efficacious therapy in the management of glaucoma drainage blebs.

Soares (Med Phys 1995, 22(9): 1487-1493) has published a paper detailing the dosimetry of typical beta ophthalmic applicators that apply a disk-shaped beta RBS to the eye. The Soares work demonstrates that, by inspection of the surface planar isodose figures, the delivered dose across the radius falls off precipitously in most of these devices. The Soares results are similar to earlier publications such as that of Bahrassa and Datta (Int J Radiat Oncol Biol Phys 1983, 9(5): 679-84), which discloses that for a typical applicator, the dose at 3.5 mm from the center-point is only 50% of the center-point maximum dose (see FIG. 1).

Generally, in legacy beta applicators, it seems that about 90% of the central maximum dose falls only within about the inner 2 mm diameter of the applicator disk. The dose appears to be reduced significantly further out along the diameter of the applicator disk. This relative under-dosing of the periphery is a significant portion of the total irradiated surface area and a significant portion of the volume of the irradiated target tissue. In addition, Soares also showed irregular dosage patterns and large variation between even the same model applicator (Soares observed that previously made beta radiation sources did not provide a uniform dose profile across the target area). Many of the applicators did not seem to have the maximum dose active portion aligned with the center of the applicator.

Safety concerns led to the narrowing of the therapeutic area in ophthalmic applicators used for pterygium treatment by attaching field-shaping masks with the effect of providing a narrowed focal application. In 1956, Castroviejo (Trans Am Acad Ophthalmol Otolaryngol. 1956, 60(3):486) introduced a series of four screening masks designed to fit snugly over the end of the applicator. The masks are constructed of stainless steel 0.5 mm thick that substantially blocks the beta radiation and thus restricts irradiation to only that area of the cut out of the masks. These are used to reduce the active surface area of the applicator. The masks are supplied with circular areas of 3 mm or 5 mm in diameter, and elongated areas 2 mm and 3 mm wide and 8.7 mm long. The masks blocked approximately 93% of the radiation, limiting the emission of radiation to a small amount directed to a very small area (with respect to the overall surface area of the cap, e.g., radiation attenuation mask). The Castroviejo masks do not provide a uniform dose over the total area of the disk applicator. Thus, the previous art of the Castroviejo Masks is not effective for the application of irradiation of glaucoma drainage blebs.

### Systems, Devices, and Methods of the Present Invention

The present invention features systems, devices, and methods, e.g., radionuclide brachytherapy sources (RBSs), RBS systems, brachytherapy systems and devices, and methods of use, e.g., for applying radiation to a target, e.g., a treatment area. For example, the systems and devices herein may be used to apply beta radiation to a target area in the eye to help maintain functioning blebs and/or drainage holes arising from glaucoma drainage procedures or surgeries, to help avoid scar formation or wound reversion, to inhibit or reduce fibrogenesis and/or inflammation in the blebs or surrounding areas, etc.

The present invention provides the unique technical features of the use of beta radiation instead of, or in combination with, antimetabolites in treatment of conjunctival blebs for prophylaxis and/or treatment of scar.

The present invention also provides the unique technical feature of the delivery of a more optimized dose distribution across the surface of the treatment area (target area) and/or a surface or plane within the treatment area (target area) as compared to devices previously used.

Without wishing to limit the present invention to any theory or mechanism, in certain embodiments, the term “optimized dose distribution” or “uniform dose” may refer to a dose within an area of a particular plane (e.g., the plane of the surface of the source or system (e.g., source with radiation attenuation mask), a plane on or within the target area or treatment area, etc.) that is substantially uniform wherein the doses within that area of the particular plane vary by no more than a certain percentage of the maximum dose, e.g, the average maximum dose of the particular plane, the average maximum dose of the treatment volume, etc. The present invention is not limited to a particular size of the area of the particular plane with substantially uniform doses, nor any particular dimensions of the target and depths of the target plane.

In certain embodiments, the term “optimized dose distribution” may also mean that the dose distribution is shaped across the lesion in a specific pattern with the intention to best affect the therapeutic outcome. In one example, the dose distribution across the diameter/plane at the treatment depth may vary such that the areas at the edges of the bleb receive a higher dose relative to the center. In one example, the dose distribution across the diameter/plane at the treatment depth may vary such that the area at a MIGS device outflow orifice receives a boosted dose compared to other areas. In one example, the dose distribution across the diameter/plane at the treatment depth may vary such that the edges of the bleb and also the area at the MIGS device outflow orifice both receive a boosted dose. In one example, the dose is attenuated over a specified area. In one example, the dose is attenuated over the cornea.

Beta radiation attenuates quickly with depth. In some embodiments, the term “optimized dose distribution” includes an appropriate dose through the depth of the target tissue. The clinical dosage depth may be determined by the thickness of the conjunctiva and associated tenon's capsule of a functional bleb. For MIGS surgery, the focus area may be approximately 3 mm above the superior limbus. Howlet et al., found the mean thickness of the conjunctival and Tenon's layer to be 393±67 microns ranging from 194 to 573 microns using optical coherence tomography (OCT) in glaucoma patients (Howlet J et al., Journal of Current Glaucoma Practice 2014, 8(s):63-66). In an earlier study, Zhang et al. found conjunctival thickness to be 238±51 microns in healthy individuals using OCT analysis and concluded OCT accurately measures the cross-sectional structures of conjunctival tissue with high resolution (Zhang et al., Investigative Ophthalmology & Visual Science 2011, 52(10):7787-7791). Based on the Howlet study, the target tissue thickness may range from 150 to 700 microns, or from 10 to 700 microns, etc. In one example, the dose distribution from the surface through the depth of the target tissue allows for a therapeutic dose within the tissue to the limits of the rapidly attenuating beta rays.

### SUMMARY OF THE INVENTION

It has been surprisingly discovered that specific methods of treatment and systems that combine Minimally Invasive Glaucoma Surgery (MIGS) implants or Minimally Invasive Micro Sclerostomy (MIMS) or the like and the application of beta radiation are effective for maintaining functioning drainage blebs, e.g., by reducing or inhibiting foreign body induced scar formation or wound revision, by inhibiting or reducing fibrogenesis and/or inflammation in the bleb, etc. The use of beta radiation in trabeculectomy-type glaucoma treatment has long been discouraged by experts in the field. However, beta radiation has been found to be surprisingly effective at preventing bleb failure when combined with use of MIGS implants or MIMS procedures.

### Unpredictability of the Effects of Beta Radiation on MIGS Implant Foreign Body Induced Scarring Response or MIMS-Induced Scarring Response

There has been no evidence to prove that the scarring responses caused by trabeculectomy surgery and MIGS implantation or MIMS are the same. In fact, there is a strong suggestion that the responses could be significantly different. Therefore, a person having ordinary skill in the art would not be able to predict how beta radiation would affect the scarring responses caused by a MIGS implant or MIMS procedure.

For example, because a MIGS implant is a foreign body implanted within the eye, there is a question of how the biocompatibility of the implant affects the scarring response. As stated in a study comparing different biomaterials for glaucoma drainage devices, “The inflammatory response following the implantation of different biomaterials in the subconjunctival space may vary and could contribute to the success or failure of the operation,” (Ayyala et. al., Arch Ophthalmol. 1999; 117:233-236). A biocompatibility study on the InnFocus MicroShunt® implant noted: “It is believed that the fibrotic and inflammatory reactions induced by biomaterials are a major determinant of success. Other factors such as shape, flexibility, modulus, and texture could also be associated with erosion, extrusion, inflammation, and scarring,” (Acosta et. al., Arch Opthalmol. 2006; 124; 1742-1749).

An additional example of the well-documented issue of the foreign body reaction states, “[Glaucoma filtration surgery] often fails because of scarring. Various conjunctival implants have been developed to minimize the scarring but may cause a foreign body reaction and capsule formation resulting in reduced efficacy and sub-optimal pharmacokinetics,” (Khaw et. al., 2015, ARVO Poster Abstract).

Because of the industry expectation that the scarring responses to trabeculectomy surgery (wherein a foreign body is not implanted) and MIGS (wherein a foreign body is implanted) or MIMS (wherein a drainage channel is formed) would not be the same, it is impossible to predict how beta radiation would affect scarring from MIGS device implantation.

Teaching Away from the Use of Beta Radiation for Glaucoma Treatment
1. Industry Expectation that Mitomycin C (MMC) is More Effective than Beta Radiation:

It would be surprising to one of ordinary skill in the art that beta radiation would be chosen over liquid antimetabolites because the prior art teaches that beta radiation is a less effective anti-metabolite than mitomycin C (MMC) and is merely similar in effectiveness to 5-fluorouracil (5FU). In brief, beta radiation has been reported to be roughly equivalent to 5FU as an anti-metabolite for glaucoma drainage surgery, and MMC has been reported to be superior to 5FU for the same use. Therefore, MMC is taught to be more effective than beta radiation as an anti-metabolite for glaucoma drainage surgery. More specifically, a 2016 study involving a trabeculectomy-type of glaucoma surgery (Dhalla et al., 2016, PLoS ONE 11(9): e0161674) concluded that: “Firstly, there is no evidence of a difference between the use of 5FU and beta radiation as an anti-metabolite in phacotrabeculectomy surgery.” Additionally, a 2015 Cochrane review by Cabourne et al. (Cabourne et al., 2015, Cochrane Database of Systematic Reviews Issue 11. Art. No.: CD006259) that compared MMC and 5FU for wound healing in trabeculectomy-type glaucoma surgery concluded: “Our review showed that the risk of failure of trabeculectomy at one year after surgery was lower in those participants treated with MMC compared to those treated with 5-FU.” Thus, since the effectiveness of beta radiation with trabeculectomy procedures is shown to be similar to that of 5FU and 5FU is shown to have inferior effectiveness compared to MMC, the literature teaches that MMC is a more effective anti-metabolite than beta radiation.

Furthermore, a direct comparative study of intraoperative mitomycin C (MMC) and beta radiation use in pterygium surgery indicated that, “intraoperative mitomycin C is more effective than β irradiation as an adjunctive treatment for pterygium surgery using a sliding conjunctival flap,” (Amano et al., 2000, British Journal of Ophthalmology 84:618-621). Thus, the prior art teaches away from use of beta radiation and instead teaches that MMC is a more effective anti-metabolite.

2. Industry Expectation that Mitomycin C (MMC) Provides More Comprehensive Penetration than Beta Radiation:

Secondly, it is surprising to use beta radiation instead of liquid antimetabolites because the prior art teaches that liquid antimetabolites are better suited for dispersion across a wide treatment area. The importance of this wide treatment area is highlighted in the Moorfields Safe Surgery System, which was developed by Sir. Peng Khaw (Khaw et al., 2005, Glaucoma Today, March/April, 22-29). The publication that introduced the System notes that previous focal treatment with MMC led to “a thin, cystic bleb.” One of the key components of the improved System is to treat “as large of an area as possible” with MMC. Critically, the publication notes: “Enlarging the surface area of treatment [with MMC] results in a more diffuse, non-cystic area, clinically. It also prevents the development of the ring of steel, which would otherwise restrict aqueous flow and promote the development of a raised, cystic, avascular bleb.”

In stark contrast to the freely flowing and widely dispersed liquid antimetabolites, the use of beta radiation for ophthalmic applications has traditionally been extremely focused. Because reproducible dosage requires that the applicator be held in place for a specified period of time, the treatment area is set by the size of the applicator head. The typical diameter of an ophthalmic applicator head is only in the order of 10-14 mm and only a fraction of the head comprises the active diameter (reported to range from 4.3 to 8.9 mm) (Soares, 1995, Med. Phys. 22 (9), September, 1487-93). Even within the active diameter, the intensity of the dose falls off quickly with increasing distance from the center of the dose.

Additionally, beta radiation is unable to effectively penetrate tissue and is restricted to treatment of superficial areas close to the center of the applicator. This is because the intensity of the dose falls off very quickly with increased distance from the applicator. For example: “[Beta radiation] is applied during the operation using a radioactive applicator which emits beta rays which have only a very local penetration to a depth of less than one millimeter,” (Kirwan et al, 2012, Cochrane Database of Systematic Reviews Art. No.: CD003433).

Testing of ophthalmic applicators in Soares et al. showed irregular dosage patterns and large variation between even the same model applicator. Many of the applicators did not even have the active portion aligned with the center of the applicator. Further, safety concerns led to the narrowing of the therapeutic area in ophthalmic applicators used for pterygium treatment by attaching a Castroviejo field-shaping masks. The effect of these masks is to provide a narrowed focal application like the one taught away from by the Moorfields Safe Surgery System. The Moorfields Safe Surgery System is considered to be a standard of care.

Thus, while antimetabolites such as MMC are freely flowing liquid solutions that can disperse across a wide area, treatment by beta radiation has been much more focally limited. The current teaching is that wide dispersion may be important for formation of a healthy diffuse bleb. Beta radiation does not have the ability to fluidly disperse across the tissue in the same manner as MMC. This limitation would prevent one having ordinary skill in the art from envisaging beta radiation as being able to effectively treat the wide area currently treated by permeation with liquid antimetabolites (or the deep hole created by a MIMS procedure). Thus, the prior art teaches away from use of beta radiation and instead teaches that liquid anti-metabolites provide a more pervasive and desirable treatment. It is surprising to use a therapeutic approach that has long been associated with focal application, instead of an easily dispersed liquid.

3. Industry Fear that Beta Radiation is Associated with Cataracts:

Thirdly, it is surprising to use beta radiation instead of liquid antimetabolites because of a long history of reported correlation between beta radiation and cataracts. Beta radiation has been avoided in glaucoma treatment because of the widely held belief by leading ophthalmologists that beta radiation would cause cataracts. For example, a 2012 Cochrane review (Kirwan et al, 2012, Cochrane Database of Systematic Reviews Art. No.: CD003433) on four trials that randomized 551 people, entitled Beta Radiation for Glaucoma Surgery, concluded that “people who had beta irradiation had an increased risk of cataract after surgery.” As an additional example: Merriam et al concluded that the minimum cataractogenic dose for a single treatment was 200 cGy to the lens epithelium, with the probability of cataract approaching unity for a dose of 750 cGy (see Merriam G R, 1965, Trans Am Ophthalmol Soc. 54: 611-653, summarized by Kirwan et al, Eye (2003) 17, 207-215. doi:10.1038/sj.eye.6700306). The literature has made clear that the medical community teaches to avoid treatment of glaucoma with beta radiation.

In the same 2003 review on beta radiation, Kirwan also described some of the negative study reports regarding use of beta radiation in ophthalmology. The review emphasized that: “Adverse effects with beta radiation for pterygium have been widely reported. Earlier reports concentrated on lens opacity, conjunctival telangectasia, and other side effects of doses much higher than those used clinically after pterygium surgery,” and that “Use of beta radiation for pterygium has diminished, with conjunctival autografting and topical mitomycin C now being widely used.” Furthermore, in addition to the adverse effects noted by others, Kirwan also later reported adverse effects in his own study on the use of beta radiation for the treatment of trabeculectomy patients.

The powered, controlled and randomized study on the effect of beta radiation on success of trabeculectomy-type glaucoma surgery was published by Kirwan in 2006. Notably, the study demonstrated that, “an increased risk for cataract surgery (a known complication of trabeculectomy) in the beta radiation arm during the two years after surgery.” At two years after the study the risk of developing a cataract requiring extraction was 16.7% in the radiation group and only 3.2% in the placebo group. Kirwan noted, “If beta radiation increases the need for further surgery the advantages of single therapy with trabeculectomy are much diminished.”

The previously acknowledged risk and subsequent observed incidence of cataracts following the application of beta radiation was a strong discouragement against the use of beta radiation in glaucoma treatment. The randomized controlled clinical trial results revealed a notable increased incidence of cataracts associated with beta therapy; and the Kirwan authors called for an “urgent study . . . of combined surgery (trabeculectomy with beta radiation plus cataract extraction).”

Ethically-engaged research requires a commitment to universal ethical norms, such as those expressed in the Declaration of Helsinki and the Belmont Report. The World Health Organization (Research ethics committees: basic concepts for capacity-building. World Health Organization 2009) notes that research ethics committees review proposed studies with human participants to ensure that they conform to internationally and locally accepted ethical guidelines. Review by a research ethics committee is required by international ethical standards governing research involving human participants, as well as by local law in many jurisdictions. In the light of their role in identifying and evaluating the risks and benefits of research, research ethics committees must include individuals with scientific and medical expertise. In studies involving medical interventions, research ethics committees must determine that adequate care and treatment will be provided for participants.

“Research funded by the United States of America (USA) government, regardless of the setting where the research takes place, must conform to the ‘Common Rule’ (45 CFR 46) that defines and regulates the scope and review of federally-funded human subjects research,” (https://www.ncbi.nlm.nih.gov/pmc/articles/PMC3491753/0). “The International Council on Harmonisation (ICH) defines an institutional review board (IRB) as a group formally designated to protect the rights, safety and well-being of humans involved in a clinical trial by reviewing all aspects of the trial and approving its startup. IRBs can also be called independent ethics committees (IECs). An IRB/IEC reviews the appropriateness of the clinical trial protocol as well as the risks and benefits to study participants. RB/IEC members should be collectively qualified to review the scientific, medical and ethical aspects of the trial. An IRB/IEC should have . . . at least five members . . . Competent members who are able to review and evaluate the science, medical aspect and ethics of the proposed trial,” (http://www.ppdi.com/Participate-In-Clinical-Trials/Become-an-Investigator/Institutional-R eview-Board). The US 21 CFR Part 56 (22)(c) declares that “Institutional Review Board (IRB) means any board, committee, or other group formally designated by an institution to review, to approve the initiation of, and to conduct periodic review of, biomedical research involving human subjects. The primary purpose of such review is to assure the protection of the rights and welfare of the human subjects.” Both international norms, and in the US, US 21 CFR Part 56 Sec. 56.107 mandate that “Each IRB shall have at least five members . . . possessing the professional competence necessary to review the specific research activities, the IRB shall be able to ascertain the acceptability of proposed research in terms of institutional commitments and regulations, applicable law, and standards of professional conduct and practice.” “In Grimes v. Kennedy Krieger Institute, Inc., a Maryland state court, although it has been criticized for doing so, honored the Nuremberg Code as “the most complete and authoritative statement of the law of informed consent to human experimentation,” (emphasis added). The court then goes on to cite several authors to support what appears to be its general premise, that the Nuremberg Code should be incorporated into American common law jurisprudence to establish a clear set of duties as to the protection offered the subjects of human experimentation,” (https://www.ncbi.nlm.nih.gov/pmc/articles/PMC1069025).

Following the findings of the Kirwan study of increased cataract in the beta therapy patient group, Dhalla studied the concomitant treatment regimen of beta therapy with phacoemulsification. The Dhalla human clinical study surgically removed the patients' natural lenses at the time of beta administration. The study authors argue that this protocol is ethical even in those patients in which “if the [pre-existing] cataract does not cause significant disability it would not normally warrant surgical intervention.” In other words, under normal conditions these patients would not be offered cataract surgery because the local standard of care would not warrant surgical intervention. The Dhalla beta therapy protocol included the additional surgical intervention of removing the patients' natural lens because the Kirwan study findings of increased incidence of cataract with the use of beta radiation alone.

In approving the Dhalla experimental study as ethical, the human study independent ethics committee decision provides direct authoritative teaching away from the use of beta therapy as a stand-alone adjunct to glaucoma filtration surgery.

Note that the outcome results of the Dhalla experimental human study were negative. “[The] study sample size calculation was based on detecting superiority of beta-radiation over 5FU [5 fluorouracil] which was the standard treatment . . . . We detected no major difference between 5 fluorouracil and beta radiation.” The disappointing study outcomes of the Dhalla study informed the medical community that beta was not superior to the antimetabolite 5FU.

The industry expectation that antimetabolites such as 5FU and MMC are more effective than beta radiation, combined with the expectation that 5FU and MMC provide more comprehensive penetration than beta radiation and the fear that beta radiation is associated with cataracts, strongly teaches away from the use of beta radiation. Thus, it would be surprising to one having ordinary skill in the art to use beta radiation with MIGS implants or MIMS to maintain functioning drainage blebs for the treatment of glaucoma.

### Brief Summary of Particular Embodiments

The present invention provides radionuclide brachytherapy sources (RBS) comprising a beta radioisotope component encased in a capsule wherein at least a portion of a bottom surface of the RBS is an active area, the active area being where beta radiation is emitted from, wherein an area within 3-4 mm from the center of the active area of the bottom surface of the RBS emits a dose that is at least 80% of the dose emitted at the center of the active area.

In some embodiments, the RBS is cylindrical. In some embodiments, the RBS is oval-shaped, non-cylindrical kidney-shaped, rectangular, elliptical, triangular, or irregular in shape. In some embodiments, the beta radioisotope component is in a disc configuration. In some embodiments, the beta radioisotope component is in an annulus configuration. In some embodiments, the beta radioisotope component is oval shaped, non-cylindrical kidney-shaped, rectangular, elliptical, triangular, or irregular in shape.

In some embodiments, the RBS has a diameter from 2 to 20 mm. In some embodiments, the RBS has a diameter from 4 to 15 mm. In some embodiments, the active area is from 6 to 12 mm in diameter. In some embodiments, the active area is from 7 to 10 mm in diameter.

In some embodiments, the encasement is constructed from a material comprising stainless steel, gold, platinum, titanium, tantalum, titanium alloy, silver, tin, zinc, copper, nickel, aluminum, a ceramic, glass, a metal alloy, zirconium, or a combination thereof.

In some embodiments, the beta radioisotope comprises Strontium-90 (Sr-90). In some embodiments, the beta radioisotope comprises Yttrium 90 (Y-90). In some embodiments, the beta radioisotope comprises Strontium-90 in secular equilibrium with Yttrium 90.

The present invention also provides an RBS system comprising an RBS according to the embodiments of the present invention, and a radiation attenuation mask for housing a radionuclide brachytherapy source (RBS), the radiation attenuation mask comprises a side wall, an open top end for accepting the RBS, and a bottom end sealed with the side wall along an entire bottom circumference of the side wall, forming an inner cavity.

In some embodiments, the radiation attenuation mask further comprises an attenuation component disposed in the inner cavity on the bottom surface, the dose flattening filter prevents a portion of beta radiation emitted from an active area of the RBS from passing through the bottom surface, thereby controlling an amount of beta radiation emitted from the bottom surface of the radiation attenuation mask.

In some embodiments, the radiation attenuation mask is cylindrical. In some embodiments, the component is annulus shaped. In some embodiments, the component is dome-shaped. In some embodiments, the component is integrated into the bottom surface of the radiation attenuation mask.

In some embodiments, the bottom surface of the radiation attenuation mask is 12 mm in diameter. In some embodiments, the bottom surface of the radiation attenuation mask is from 8 to 10 mm in diameter. In some embodiments, the bottom surface of the radiation attenuation mask is from 10 to 12 mm in diameter. In some embodiments, the bottom surface of the radiation attenuation mask is from 7 to 14 mm in diameter.

The present invention also features a brachytherapy system for applying beta radiation to a target plane of a treatment area, said system comprises a radionuclide brachytherapy source (RBS) according to the embodiments of the present invention, and a radiation attenuation mask according to the embodiments of the present invention.

In some embodiments, the radiation attenuation mask is attachable to a brachytherapy applicator handle, the RBS is sandwiched between the radiation attenuation mask and the handle.

In some embodiments, the system is for single use. In some embodiments, one or more components of the system can be sterilized.

In some embodiments, the system is for treating glaucoma. In some embodiments, the system is for reducing intraocular pressure (IOP). In some embodiments, the system is for treating glaucoma-associated drainage blebs. In some embodiments, the system is for reducing or inhibiting fibrogenesis or inflammation in a bleb or a drainage hole.

The present invention also includes an RBS system having a dose profile wherein all points of a treatment volume within a 4 mm radius has a dose that is at least 80% of the dose at the center, wherein the points of the treatment volume are at a depth relative to the RBS system. In some embodiments, the depth is 0.2 mm. In some embodiments, the depth is 0.2 to 1 mm. In some embodiments, the depth is 1 mm. In some embodiments, the depth is from 1 to 1.5 mm. In some embodiments, the depth is 2 mm. In some embodiments, the dose profile is therapeutic. In some embodiments, the target is a target in an eye. In some embodiments, all points within an area of 4 mm from the center emits a dose that is at least 80% of the dose at the center. In some embodiments, all points within an area of 4 mm from the center emits a dose that is at least 80% of the dose at the center. In some embodiments, all points within an area of 4 mm from the center emits a dose that is at least 80% of the dose at the center.

The present invention also features a method of inhibiting or reducing fibrogenesis and inflammation in a bleb of an eye being treated for glaucoma, wherein a Minimally Invasive Glaucoma Surgery (MIGS) implant is inserted trans-sclerally and causes formation of a bleb in the subconjunctival space of the eye or in a space between the conjunctiva and Tenon's capsule. In some embodiments, the method comprises applying a therapeutic amount of beta radiation from a radioisotope to a target area of the eye using an applicator system as described herein, e.g., an RBS and/or attenuation mask and/or applicator according to the present invention. In some embodiments, the bottom surface of the radiation attenuation mask is for contacting a target tissue. In some embodiments, the beta radiation causes cell cycle arrest in fibroblasts on the Tenon's capsule to inhibit or reduce the fibrotic process and inflammation that leads to bleb failure.

The present invention also features a method of maintaining a functioning drainage bleb in the eye of a patient being treated for glaucoma. In some embodiments, the method comprises implanting a Minimally Invasive Glaucoma Surgery (MIGS) implant within the eye, wherein the implant is inserted trans-sclerally and causes formation of a bleb in the subconjunctival space of the eye or in a space between the conjunctiva and Tenon's capsule, the bleb functions to drain aqueous humor; and applying a therapeutic amount of beta radiation from a radioisotope to a target area of the eye using an applicator system. In some embodiments, the applicator system comprises a system according to the present invention such as an RBS and/or a radiation attenuation mask and/or an applicator. In some embodiments, the bottom surface of the radiation attenuation mask is for contacting a target tissue. In some embodiments, the beta radiation reduces or inhibits a fibrotic process and inflammation that causes bleb failure, and wherein the method is effective to maintain the drainage function of the bleb.

The present invention also features a method of treating glaucoma. In some embodiments, the method comprises implanting a Minimally Invasive Glaucoma Surgery (MIGS) implant within an eye of a patient being treated for glaucoma, wherein the implant is inserted between an anterior chamber of the eye and a subconjunctival space of the eye or between the anterior chamber of the eye and a space between the conjunctiva and Tenon's capsule, the implant causes formation of a bleb for draining aqueous humor; and applying a therapeutic amount of beta radiation from a radioisotope to a target area of the eye using an applicator system according to the present invention. The method may be effective for reducing an Intraocular Pressure (IOP) of the eye.

The present invention also features a method of treating glaucoma. In some embodiments, the method comprises performing a glaucoma drainage surgery in an eye to form a bleb in a subconjunctival space or between the conjunctiva and Tenon's capsule and to allow aqueous humor to drain into the drainage bleb; and applying a modified therapeutic amount of beta radiation from a radionuclide brachytherapy (RBS) system to a target area associated with the bleb, a drainage channel, a drainage implant, or a combination thereof. In some embodiments, the target area has a diameter of 8 mm. In some embodiments, the method is effective for lowering intraocular pressure (IOP).

The present invention also features a method of reducing intraocular pressure (IOP) in an eye. In some embodiments, the method comprises implanting a Minimally Invasive Glaucoma Surgery (MIGS) implant within an eye of a patient being treated for glaucoma, wherein the implant is inserted between an anterior chamber of the eye and a subconjunctival space of the eye or between the anterior chamber of the eye and a space between the conjunctiva and Tenon's capsule, the implant causes formation of a bleb for draining aqueous humor; and applying a therapeutic amount of beta radiation from a radioisotope to a target area of the eye using an applicator system according to the present invention. In some embodiments, the beta radiation is effective for reducing an Intraocular Pressure (IOP) of the eye

The present invention also features a method of reducing intraocular pressure (IOP) in an eye being treated or having been treated with glaucoma drainage surgery to form a bleb in a subconjunctival space or between the conjunctiva and Tenon's capsule and to allow aqueous humor to drain into the drainage bleb, said method comprising applying a modified therapeutic amount of beta radiation from a radionuclide brachytherapy (RBS) system to a target area associated with the bleb, a drainage channel, a drainage implant, or a combination thereof, wherein the method is effective for lowering intraocular pressure (IOP). In some embodiments, the target has a diameter of 8 mm and a depth from 0 to 0.4 mm.

The present invention also features a method of reducing inflammation in an eye having a foreign body therein, the foreign body being a Minimally Invasive Glaucoma Surgery (MIGS) implant inserted between an anterior chamber of the eye and a subconjunctival space of the eye or between the anterior chamber of the eye and a space between the conjunctiva and Tenon's capsule, the implant causes formation of a bleb for draining aqueous humor, said method comprising: applying a therapeutic amount of beta radiation from a radioisotope to a target area of the eye using an applicator system, the applicator system comprises a system system according to the present invention. In some embodiments, the bottom surface of the radiation attenuation mask is for contacting a target tissue. In some embodiments, the method is effective for reducing inflammation caused by the presence of the foreign body.

In some embodiments, when the applicator system is placed in contact with the eye at the target tissue and pressed upon, the distance from the bottom surface of the radiation attenuation mask and the bottom surface of the bleb is substantially uniform across the target area. In some embodiments, when the applicator system is placed in contact with the eye at the target tissue and pressed upon, the distance from the bottom surface of the radiation attenuation mask and the top surface of the bleb is substantially uniform across the target area.

In some embodiments, the target is at least a portion of a bleb. In some embodiments, the target comprises an entire bleb. In some embodiments, the target comprises a portion of a bleb. In some embodiments, the target area surrounds an end of the MIGS implant. In some embodiments, the target comprises at least a portion of the bleb above a drainage channel. In some embodiments, the target further comprises at least a portion of the bleb above a drainage channel and at least a portion of a perimeter of the bleb. In some embodiments, target further comprises at least a portion of the bleb above a drainage channel, at least a portion of a perimeter of the bleb, and at least a portion of the bleb between the perimeter and the portion above the drainage channel. In some embodiments, therapeutic dose is from 500-1000 cGy. In some embodiments, therapeutic dose is from 450-1050 cGy. In some embodiments, therapeutic dose is from 400-1200 cGy. In some embodiments, method further comprises administering a drug to the target area.

In some embodiments, glaucoma drainage surgery is Minimally Invasive Glaucoma Surgery (MIGS). In some embodiments, glaucoma drainage surgery is Minimally Invasive Micro Sclerostomy (MIMS). In some embodiments, glaucoma drainage surgery is trabeculectomy.

In some embodiments, the therapeutic amount of beta radiation helps maintain a functioning drainage bleb. In some embodiments, the therapeutic amount of beta radiation helps reduce conjunctival inflammation. In some embodiments, the radioisotope comprises Strontium-90 (Sr-90), Phosphorus-32 (P-32), Ruthenium 106 (Ru-106), Yttrium 90 (Y-90), or a combination thereof. In some embodiments, the therapeutic dose is from 500-1000 cGy. In some embodiments, the therapeutic dose is from 450-1050 cGy. In some embodiments, the method further comprises administering a drug to the target area.

In some embodiments, the target is at least a portion of a bleb. In some embodiments, the target comprises an entire bleb. In some embodiments, the target comprises a portion of a bleb. In some embodiments, the target area comprises an end of a Minimally Invasive Glaucoma Surgery (MIGS) implant.

In some embodiments, the method is effective for reducing IOP to 18 mmHg or less. In some embodiments, the method is effective for reducing IOP to 15 mmHg or less. In some embodiments, the method is effective for reducing IOP to 12 mmHg or less. In some embodiments, the method is effective for reducing IOP to 10 mmHg or less. In some embodiments, the method is effective for reducing IOP and subsequent stabilization of said IOP.

In some embodiments, the method is effective for one or a combination of: maintaining a functioning drainage bleb; inhibiting or reducing fibrogenesis and inflammation in the bleb, around the drainage implant, or around the drainage channel; and reducing conjunctival inflammation in the eye.

In some embodiments, inhibiting or reducing fibrogenesis and inflammation in the bleb is measured according to a predetermined bleb grading scale. In some embodiments, the predetermined bleb grading scale is Moorfields bleb grading scale (MBGS). In some embodiments, the predetermined bleb grading scale is Indiana Bleb Appearance Grading Scale (IBAGS).

In some embodiments, the dose of beta radiation is applied using fractionation. In some embodiments, the beta radiation is rotated at least 1 time during application. In some embodiments, the beta radiation is rotated at least 2 times during application. In some embodiments, the beta radiation is rotated at least 3 times during application. In some embodiments, the beta radiation is rotated 4 times during application.

In some embodiments, the target area is at a depth of 0.5 mm. In some embodiments, the target area is at a depth of 0.7 mm. In some embodiments, the target area is at a depth of 1 mm.

The present invention also features a radionuclide brachytherapy source (RBS) system that emits beta radiation for use in a method of treating glaucoma. In some embodiments, the method comprises performing a glaucoma drainage surgery on an eye of a patient to form a bleb in a subconjunctival space or between the conjunctiva and Tenon's capsule, and to allow aqueous humor to drain into the bleb; and applying a modified therapeutic dose of beta radiation from the RBS system according to the present invention to a target area associated with the bleb, a drainage channel, a drainage implant, or a combination thereof. In some embodiments, the method is effective for lowering intraocular pressure (IOP). In some embodiments, the target area has a diameter of 8 mm. In some embodiments, the target area has a diameter of 10 mm. In some embodiments, the target has a depth from 0 to 0.4 mm.

The present invention also features a radionuclide brachytherapy source (RBS) system that emits beta radiation for use in a method of treating glaucoma in an eye of a patient having been treated with glaucoma drainage surgery wherein a bleb was formed in a subconjunctival space or between the conjunctiva and Tenon's capsule so as to allow aqueous humor to drain into the bleb. In some embodiments, the method comprises applying a modified therapeutic dose of beta radiation from the RBS system according to the present invention to a target area associated with the bleb, a drainage channel, a drainage implant, or a combination thereof. In some embodiments, the target area has a diameter of 8 mm. In some embodiments, the target area has a diameter of 10 mm. In some embodiments, the target has a depth from 0 to 0.4 mm.

The present invention also features a radionuclide brachytherapy source (RBS) system that emits beta radiation for use in a method of treating glaucoma. In some embodiments, the method comprises performing a glaucoma drainage surgery on an eye of a patient to form a bleb in a suboonjunctival space or between the conjunctiva and Tenon's capsule, and to allow aqueous humor to drain into the bleb; and applying a modified therapeutic dose of beta radiation from the RBS system (e.g., an RBS system according to the present invention) to a target area associated with the bleb, a drainage channel, a drainage implant, or a combination thereof. In some embodiments, the target area has a diameter of 8 mm. In some embodiments, the target area has a diameter of 10 mm. In some embodiments, the target has a depth from 0 to 0.4 mm.

The present invention also features a radionuclide brachytherapy source (RBS) system that emits beta radiation for use in a method of treating glaucoma in an eye of a patient having been treated with glaucoma drainage surgery wherein a bleb was formed in a suboonjunctival space or between the conjunctiva and Tenon's capsule so as to allow aqueous humor to drain into the bleb. In some embodiments, the method comprises applying a modified therapeutic dose of beta radiation from the RBS system (e.g., an RBS system according to the present invention) to a target area associated with the bleb, a drainage channel, a drainage implant, or a combination thereof. In some embodiments, the target area has a diameter of 8 mm. In some embodiments, the target area has a diameter of 10 mm. In some embodiments, the target has a depth from 0 to 0.4 mm.

The present invention also features a radionuclide brachytherapy source (RBS) system that emits beta radiation for use in a method of treating glaucoma. In some embodiments, the method comprises performing a glaucoma drainage surgery on an eye of a patient to form a bleb in a suboonjunctival space or between the conjunctiva and Tenon's capsule, and to allow aqueous humor to drain into the bleb; and applying a modified therapeutic dose of beta radiation from the RBS system (e.g., an RBS system according to the present invention) to a target area associated with the bleb, a drainage channel, a drainage implant, or a combination thereof. In some embodiments, the target area has a diameter of 8 mm. In some embodiments, the target area has a diameter of 10 mm. In some embodiments, the target has a depth from 0 to 0.4 mm.

The present invention also features a radionuclide brachytherapy source (RBS) system that emits beta radiation for use in a method of treating glaucoma in an eye of a patient having been treated with glaucoma drainage surgery wherein a bleb was formed in a subconjunctival space or between the conjunctiva and Tenon's capsule so as to allow aqueous humor to drain into the bleb. In some embodiments, the method comprises applying a modified therapeutic dose of beta radiation from the RBS system (e.g., an RBS system according to the present invention) to a target area associated with the bleb, a drainage channel, a drainage implant, or a combination thereof. In some embodiments, the target area has a diameter of 8 mm. In some embodiments, the target area has a diameter of 10 mm. In some embodiments, the target has a depth from 0 to 0.4 mm.

In some embodiments, a target area having a depth of 0 is in contact with the RBS system.

The present invention also features kits. In certain embodiments, the kit comprises a radiation attenuation mask according to the present invention; a radionuclide brachytherapy source according to the present invention; and a handle (e.g., applicator) attachable to the radiation attenuation mask to secure the radionuclide brachytherapy source in the radiation attenuation mask. In certain embodiments, the kit comprises a radiation attenuation mask according to the present invention; and a handle (e.g., applicator) attachable to the radiation attenuation mask to secure the radionuclide brachytherapy source in the radiation attenuation mask. In certain embodiments, one or more components of the kit are provided in sterile packaging. In certain embodiments, the kit is for single use.

The RBS of the present invention is constructed in a manner that is consistent with the Federal Code of Regulations, but is not limited to the terms mentioned in the Code. For example, the RBS of the present invention may further comprise a substrate. Also, for example, in addition to being enclosed by the mentioned “gold, titanium, stainless steel, or platinum”, in some embodiments the radionuclide (isotope) of the present invention may be enclosed by a combination of one or more of “gold, titanium, stainless steel, or platinum”. In some embodiments, the radionuclide (isotope) of the present invention may be enclosed by one or more layers of an inert material comprising silver, gold, titanium, stainless steel, platinum, tin, zinc, nickel, copper, other metals, ceramics, glass, or a combination of these.

In some embodiments, the RBS comprises a substrate, a radioactive isotope (e.g., Sr-90, Y-90, Rh-106, P-32, isotopes of cesium, 1-125, etc.), and a capsule or an encapsulation. In some embodiments, the isotope is coated on the substrate, and both the substrate and isotope are further coated with the encapsulation. In some embodiments, the radioactive isotope is embedded in the substrate. In some embodiments, the radioactive isotope is part of the substrate matrix. In some embodiments, the encapsulation may be coated onto the isotope, and optionally, a portion of the substrate. In some embodiments, the encapsulation is coated around the entire substrate and the isotope. In some embodiments, the encapsulation encloses the isotope. In some embodiments, the encapsulation encloses the entire substrate and the isotope. In some embodiments, the radioactive isotope is an independent piece and is sandwiched between the encapsulation and the substrate. The substrate may be constructed from a variety of materials. For example, in some embodiments the substrate is constructed from a material comprising, a silver, an aluminum, a stainless steel, tungsten, nickel, tin, zirconium, zinc, copper, a metallic material, a ceramic material, a ceramic matrix, the like, or a combination thereof. In some embodiments, the substrate functions to shield a portion of the radiation emitted from the isotope. The encapsulation may be constructed from a variety of materials, for example from one or more layers of an inert material comprising a steel, a silver, a gold, a titanium, a platinum, another biocompatible material, the like, or a combination thereof.

The systems herein are constructed to provide a substantially uniform radiation dose across the target, e.g., across a particular diameter of a target volume and through a particular depth of the target volume. Relative to other sources, the present invention may provide a more uniform dose across the target area, e.g., the center is not overdosed. The present invention is not limited to the dosimetry profiles shown or described herein.

In some embodiments, one or more components of the invention (e.g., cap) are constructed from a material that can further shield the user from the RBS. In some embodiments, a material having a low atomic number (Z) may be used for shielding (e.g., polymethyl methacrylate). In some embodiments, a material having a high atomic number (Z) may be used for shielding (e.g., iron alloys, gold, lead, tungsten, tantalum, etc.). In some embodiments, one or more layers of material are used for shielding, wherein one comprises a material having a low atomic number and another layer having a higher atomic number. In some embodiments, one or more layers of material are used for shielding, wherein an outer layer comprises a material having a low atomic number (e.g., high impact polystyrene (HIPS)) and is biocompatible and sterilizable, and an inner layer having a HIGH atomic number. In some embodiments, the shielding contains high Z materials suspended in a low Z polymer. In some embodiments, the shielding contains tungsten powder suspended or formed in a polymer carrier.

The present invention features brachytherapy systems for applying radiation to a target area. While the present invention describes applications of the systems and devices for treating glaucoma drainage bleb tissues or drainage holes, e.g., to help avoid scar formation or wound reversion, to inhibit or reduce fibrogenesis and/or inflammation in blebs or holes, etc., the present invention is not limited to the applications disclosed herein.

As used herein, the dose may be defined as the dose in water or the dose in water equivalent plastic (e.g., plastic water), or the dose in tissue.

The present invention also features a radionuclide brachytherapy source (RBS) comprising a beta radioisotope component encased in a capsule wherein at least a portion of a bottom surface of the RBS is an active area, the active area being where beta radiation is emitted from, wherein an area within 3-4 mm from the center of the active area of the bottom surface of the RBS emits a dose that is at least 80% of the dose emitted at the center of the active area. In some embodiments, the RBS is cylindrical. In some embodiments, the beta radioisotope component is in a disc configuration. In some embodiments, the beta radioisotope component is in an annulus configuration.

In some embodiments, the RBS has a diameter from 4 to 15 mm. In some embodiments, the active area is from 6 to 12 mm in diameter. In some embodiments, the beta radioisotope comprises Strontium-90 (Sr-90). In some embodiments, the beta radioisotope comprises Yttrium 90 (Y-90). In some embodiments, the beta radioisotope comprises Strontium-90 in secular equilibrium with Yttrium 90.

In some embodiments, the RBS further comprises a radiation attenuation mask for housing a radionuclide brachytherapy source (RBS), wherein the radiation attenuation mask comprises a side wall, an open top end for accepting the RBS, and a bottom end sealed with the side wall along an entire bottom circumference of the side wall, forming an inner cavity.

In some embodiments, the radiation attenuation mask further comprises an attenuation component disposed in the inner cavity on the bottom surface, the dose flattening filter prevents a portion of beta radiation emitted from an active area of the RBS from passing through the bottom surface, thereby controlling an amount of beta radiation emitted from the bottom surface of the radiation attenuation mask. In some embodiments, the radiation attenuation mask is cylindrical. In some embodiments, the attenuation component is annulus shaped. In some embodiments, the attenuation component is dome-shaped. In some embodiments, the bottom surface of the radiation attenuation mask is from 7 to 14 mm in diameter.

In some embodiments, the system has a dose profile wherein all points of a treatment volume within a 4 mm radius has a dose that is at least 80% of the dose at the center, the points of the treatment volume being at a depth relative to the RBS system. In some embodiments, the depth is 0.2 to 1 mm. In some embodiments, the depth is from 1 to 1.5 mm. In some embodiments, the dose profile is therapeutic. In some embodiments, the target is a target in an eye.

The present invention also features a radionuclide brachytherapy source (RBS) comprising an active beta radioisotope material encased in a capsule, wherein the active beta radioisotope material emits beta radiation through at least a portion of a distal surface of the capsule to produce a three dimensional radiation field in a planning treatment volume (PTV). In some embodiments, the planar radiation field at a depth (e.g., 1 mm depth, 1.5 m depth, etc.) from the distal surface of the capsule is within 20% of that shown and described herein. In some embodiments, the variation is based on gamma function analysis.

The present invention also features a radionuclide brachytherapy source (RBS) comprising an active beta radioisotope material encased in a capsule, wherein the active beta radioisotope material emits beta radiation through at least a portion of a distal surface of the capsule to produce a three dimensional radiation field in a planning treatment volume (PTV). In some embodiments, the planar radiation field at a depth (e.g., 1 mm depth, 1.5 m depth, etc.) from the distal surface of the capsule is within 15% of that shown and described herein. In some embodiments, the variation is based on gamma function analysis.

The present invention also features a radionuclide brachytherapy source (RBS) comprising an active beta radioisotope material encased in a capsule, wherein the active beta radioisotope material emits beta radiation through at least a portion of a distal surface of the capsule to produce a three dimensional radiation field in a planning treatment volume (PTV). In some embodiments, the planar radiation field at a depth (e.g., 1 mm depth, 1.5 m depth, etc.) from the distal surface of the capsule is within 10% of that shown and described herein. In some embodiments, the variation is based on gamma function analysis.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references

Dosimetry techniques include film dosimetry. In one example the RBS is applied to radiographic film, for example Gafchromic™ film. The dose at various depths can also be measured by placing an intervening material, such as Plastic Water™, of known thicknesses between the RBS and the film. A transmission densitometer in conjunction with a film optical density vs. dose chart, allows for the film opacity to be measured and then converted to delivered dose. Other methods include Thermoluminescent methods (TLD chips). TLD chips are small plastic chips with millimeter dimensions having a crystal lattice that absorbs ionizing radiation. Dose is always defined in a medium, e.g., water, tissue, plastic water; if not specified, it typically refers to water.

Dose variation is described as that across the diameter assuming a central point maximum dose. However, in practice it has been demonstrated that the maximum dose may be off center. Thus, a description of variation of dose across the diameter may also include the variation of dose over the area, and through the depth.

In general use in the profession of ophthalmology the term “conjunctivae” may refer to the conjunctivae in combination with the Tenon's capsule. Also, in general use in the profession of ophthalmology the term “conjunctivae” may refer to the conjunctivae alone, not including the Tenon's capsule. References herein to “conjunctivae” can include either and/or both meanings.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Beam Modification: Desirable modification in the spatial distribution of radiation (e.g., within the patient) by insertion of any material in the beam path. Beam modification increases conformity allowing a higher dose delivery to the target, while sparing more of normal tissue simultaneously. There are four main types of beam modification: (1) Shielding: To eliminate radiation dose to some special parts of the zone at which the beam is directed. In general use is the fabrication of low-melting-temperature alloy (Lipowitz metal or Cerroblend) shielding blocks that are custom made for the individual patient and used to shield normal tissue and critical organs. For example, during total body irradiation (TBI), customized shielding blocks are positioned in front of the lungs to reduce radiation dose. (2) Compensation: To allow normal dose distribution data to be applied to the treated zone, when the beam enters obliquely through the body, or where different types of tissues are present. (3) Wedge filtration: Where a special tilt in isodose curves is obtained. (4) Flattening: Where the spatial distribution of the natural beam is altered by reducing the central exposure rate relative to the peripheral. In general use is a beam flattening filter that reduces the central exposure rate relative to that near the edge of the beam. This technique is used for linear accelerators. The filter is designed so that the thickest part is in the center. These are often constructed of copper or brass.

Brachytherapy (see also Radionuclide Brachytherapy Source (RBS): According to the American Association of Physicists in Medicine (AAPM), brachytherapy is “the clinical use of small encapsulated radioactive sources at a short distance from the target volume for irradiation of malignant tumors or nonmalignant lesions.” According to the US Federal Code of Regulations, a Radionuclide Brachytherapy Source (RBS) is “a device that consists of a radionuclide what may be enclosed in a sealed container made of gold, titanium, stainless steel, or platinum and intended for medical purposes to be placed onto a body surface or into a body cavity or tissue as a source of nuclear radiation for therapy.” Other forms of brachytherapy sources are also used in practice. For example, a commercially available conformal source is a flexible, thin film made of a polymer chemically bound to Phosphorus-32 (P-32). Another product is the TheraSphere, a radiotherapy treatment for hepatocellular carcinoma (HCC) that consists of millions of microscopic, radioactive glass microspheres (20-30 micrometers in diameter) containing Yttrium-90. Other forms of brachytherapy employ x-ray generators as sources instead of radioisotopes.

Generally, in medical practice, brachytherapy can be categorized as topical or plaque brachytherapy, intracavitary, and interstitial.

Some implementations of brachytherapy employ permanently implanted RBSs. For example, in Low Dose Rate (LDR) brachytherapy for prostate cancer, a standard of care treatment, radioactive Iodine-125 RBSs are placed directly into the prostate where they remain indefinitely.

In another implementation, High Dose Rate (HDR) brachytherapy TheraSpheres are infused into the arteries that feed liver tumors. These microspheres then embolize, lodging themselves in the liver's capillaries and bathing the malignancy in high levels of yttrium-90 radiation. In both these implementations, the total dose is given by consuming the entire radioisotope.

Some other implementations of brachytherapy employ a transient placement of the RBS. For example, in after-loaded High Dose Rate (HDR) brachytherapy, very tiny plastic catheters are placed into the prostate gland, and a series of radiation treatments is given through these catheters. A computer-controlled machine pushes a single highly radioactive iridium-192 RBS into the catheters one by one for a specified dwell time at locations throughout the volume being irradiated. The catheters are then easily pulled out, and no radioactive material is left at the prostate gland.

Another example of transient placement of an RBS includes prophylactic therapy for restenosis of coronary arteries after stent implantation. This is a non-malignant condition that has been successfully treated by placing a catheter into the coronary artery, then inserting a HDR radioactive source into the catheter and holding it there for a predetermined time in order to deliver a sufficient dose to the vessel wall.

Functioning Drainage Bleb: A bleb that is effective for draining aqueous humor from the eye to reduce intraocular pressure (IOP) of the eye to an appropriate level.

Early bleb grading systems included those proposed by Kronfeld (1969), Migdal and Hitchings (1983), and Picht and Grehn (1998). Subsequent bleb grading systems identified and incorporated a graded assessment of various bleb parameters such as vascularity, height, width, microcystic changes, encystment and diffuse/demarcated zones.

There are two recently described grading systems for clinical grading of filtering surgery blebs: the Moorfields Bleb Grading System (MBGS) and the Indiana Bleb Appearance Grading Scale (IBAGS). The MBGS built upon the system used for this tele-medicine study and expanded it to include an assessment of vascularity away from the center of the bleb and a way to represent mixed-morphology blebs. In this scheme, central area (1-5), maximal area (1-5), bleb height (1-4) and subconjunctival blood (0-1) were assessed. In addition, three areas of the bleb were graded separately for vascularity, including bleb center conjunctiva, peripheral conjunctiva and non-bleb conjunctiva. Vascularity in each area was assigned a score from 1 to 5. A study found good inter-observer agreement and clinical reproducibility in the IBAGS and MBGS (Wells A P, Ashraff N N, Hall R C, et al. Comparison of two clinical bleb grading systems. Ophthalmology 2006; 113:77-83.)

The Moorfields bleb grading system was developed as the importance of bleb appearance to outcome was realized. Blebs that develop thin avascular zones are at increased risk of leakage and late hypotony as well as sight threatening bleb related infections.

The Indiana Bleb Appearance Grading Scale is a system for classifying the morphologic slit lamp appearance of filtration blebs. The Indiana Bleb Appearance Grading Scale contains a set of photographic standards illustrating a range of filtering bleb morphology selected from the slide library of the Glaucoma Service at the Indiana University Department of Ophthalmology. These standards consist of slit lamp images for grading bleb height, extent, vascularity, and leakage with the Seidel test. For grading, the morphologic appearance of the filtration bleb is assessed relative to the standard images for the 4 parameters and scored accordingly.

For reference, a failed or failing bleb may have “restricted posterior flow with the so-called ‘ring of steel’,” e.g., a ring of scar tissue or fibrosis adhering the conjunctiva to the sclera at the periphery of the bleb that restricts the flow of aqueous humor (see Dhingra S, Khaw P T. The Moorfields Safer Surgery System. Middle East African Journal of Ophthalmology. 2009; 16(3):112-115). Other attributes of failed or failing blebs may include cystic appearance and/or changes in vascularization and/or scar tissue and/or thinning of the conjunctiva overlaying the bleb and/or a tense bleb and/or other observable or measurable changes as may be included in either the Indiana Bleb Appearance Grading Scale or Moorfields Bleb Grading System. Other functional determinants of failed or failing blebs or glaucoma surgery may include increased IOP, or IOP that has not decreased sufficiently.

Planning Treatment Volume or Planning Target Volume (PTV): An area or volume that encloses all the tissue intended for irradiation. The PTV includes the clinical target volume or clinical treatment volume (CTV).

Radioactive isotope, radionuclide, radioisotope: A radioactive isotope, known as a radionuclide or radioisotope, is an element that has an unstable nucleus and emits radiation during its decay to a stable form. There may be several steps in the decay from a radioactive to a stable nucleus. There are four types of radioactive decay: alpha, beta negative, beta positive, and electron capture. Gamma rays can be emitted by the daughter nucleus in a de-excitation following the decay process. These emissions are considered ionizing radiation because they are powerful enough to liberate an electron from another atom.

Therapeutic radionuclides can occur naturally or can be artificially produced, for example by nuclear reactors or particle accelerators. Radionuclide generators are used to separate daughter isotopes from parent isotopes following natural decay.

Non-limiting examples of radioactive isotopes following one of the four decay processes are given herein: (1) Alpha decay: radium 226, americium 241; (2) Beta minus: iridium 192, cesium 137, phosphorous 32 (P-32), strontium 90 (Sr-90), yttrium 90 (Y-90), ruthenium 106, rhodium-106; (3) Beta positive: fluorine 18; (4) Electron capture: iodine 125, palladium 106. Examples of gamma emission include iridium 192 and cesium 137.

Half-life is defined as the time it takes for one-half of the atoms of a radioactive material to disintegrate. Half-lives for various radioisotopes can range from a few microseconds to billions of years.

The term activity in the radioactive-decay processes refers to the number of disintegrations per second. The units of measure for activity in a given source are the curie (Ci) and becquerel (Bq). One (1) Becquerel (Bq) is one disintegration per second. An older unit is the Curie (Ci), wherein one (1) Ci is 3.7×1010 Bq.

The term “beta radiation source” or “source of beta radiation” can refer to the term “radioisotope.” In any of the methods or compositions here, the radioisotope or source of beta radiation may comprise Strontium-90 (Sr-90), Phosphorus-32 (P-32), Ruthenium 106 (Ru-106), Yttrium 90 (Y-90), Strontium-90 in secular equilibrium with Yttrium 90, an isotope of cesium (e.g., Cs-137), 1-125, or other radionuclides, or a combination thereof. The present invention also includes sources wherein both beta and gamma are emitted. The present invention also includes sources that provide gamma radiation but behave like beta, e.g., low energy or soft x rays that behave similarly to beta (e.g, see, for example, Lee et al., 2008, Med. Phys. 35 (11) 5151-5160).

Radionuclide Brachytherapy Source (RBS) (see also Brachytherapy): According to the US Federal Code of Regulations, a Radionuclide Brachytherapy Source (RBS) is “a device that consists of a radionuclide what may be enclosed in a sealed container made of gold, titanium, stainless steel, or platinum and intended for medical purposes to be placed onto a body surface or into a body cavity or tissue as a source of nuclear radiation for therapy.” Other forms of brachytherapy sources are also used in practice. For example, a commercially available conformal source is a flexible, thin film made of a polymer chemically bound to Phosphorus-32 (P-32). Another product is the TheraSphere, a radiotherapy treatment for hepatocellular carcinoma (HCC) that consists of millions of microscopic, radioactive glass microspheres (20-30 micrometers in diameter) containing Yttrium-90. Other forms of brachytherapy employ x-ray generators as sources instead of radioisotopes.

Beta Ophthalmic Applicators utilizing RBS containing Strontium-90 in secular equilibrium with Yttrium-90 have been utilized on the surface of the eye to treat a number of diseases. One example of such treatments is that the RBS is applied with a manual brachytherapy applicator directly to the target tissue of the eye (e.g., the conjunctivae, bare sclera, or other tissues) for a dwell time to deliver the prescription dose, and then the RBS is removed from the surface of the eye.

Treat, Treatment, Treating: These terms refer to both therapeutic treatments, e.g., elimination of a disease, disorder, or condition, and prophylactic or preventative measures, e.g., preventing or slowing the development of a disease or condition, reducing at least one adverse effect or symptom of a disease, condition, or disorder, etc. Treatment may be “effective” if one or more symptoms or clinical markers are reduced as that term is defined herein. Alternatively, a treatment may be “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a particular disease, disorder, or condition, as well as those likely to develop a particular disease, disorder, or condition due to genetic susceptibility or other factors.

Gray (Gy) is the SI-derived unit of absorbed dose and specific energy (energy per unit mass). Such energies are usually associated with ionizing radiation such as gamma particles or X-rays. It is defined as the absorption of one joule of energy in the form of ionizing radiation by one kilogram of tissue. In the SI basic units, a gray is expressed as m²·s⁻².

A sievert (Sv) is the SI-derived unit of equivalent radiation dose, effective dose, and committed dose. One sievert is the amount of radiation necessary to produce the same effect on living tissue as one gray of high-penetration x-rays. Quantities that are measured in sieverts represent the biological effects of ionizing radiation. 1 sievert is the energy absorbed by one kilogram of biological tissue, which has the same effect as one gray of the absorbed dose of gamma radiation. Therefore the sievert can be expressed in terms of other SI units as 1 Sv=1 J/kg. The Nuclear Regulatory Regulations (NRC, 10 CFR) Subpart M—Reports § 35.3045 Report and notification of a medical event, states in part that, “(a) A licensee shall report any event as a medical event, except for an event that results from patient intervention, in which—(1) The administration of byproduct material or radiation from byproduct material, except permanent implant brachytherapy, results in—(i) A dose that differs from the prescribed dose or dose that would have resulted from the prescribed dosage by more than 0.05 Sv (5 rem) effective dose equivalent, 0.5 Sv (50 rem) to an organ or tissue, or 0.5 Sv (50 rem) shallow dose equivalent to the skin; and (A) The total dose delivered differs from the prescribed dose by 20 percent or more; (B) The total dosage delivered differs from the prescribed dosage by 20 percent or more or falls outside the prescribed dosage range; or (C) The fractionated dose delivered differs from the prescribed dose for a single fraction, by 50 percent or more.

As used herein, the term “annulus” or “annular” may also refer to a variation of an annulus, such as an irregular annulus or toroidal shape.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows surface dose profiles for a Sr-90 Source (from Bahrassa, 1983) (legacy dose, prior art) and a Sr-90 source of the present invention (new dose).

FIG. 2A shows a schematic view of the target area (e.g., a planning treatment volume), such as a bleb, wherein a therapeutic dose is applied throughout the width and depth of the target area.

FIG. 2B shows a schematic representation of radiation emitted through the outer surface of the distal end of the present invention.

FIG. 3A shows non-limiting examples of shapes of RBS capsules of the present invention.

FIG. 3B shows non-limiting examples of shapes of radioactive material (beta radiation source material) for the RBSs of the present invention.

FIG. 4 shows a perspective view of a brachytherapy applicator and a radiation attenuation mask, wherein the radiation attenuation mask can engage the brachytherapy applicator to house an RBS therein.

FIG. 5 shows an embodiment of a brachytherapy system wherein an RBS is engaged with a radiation attenuation mask.

FIG. 6 shows an example of a radiation dose profile. The present invention provides a dose profile such that an area within 3-4 mm from the center gets a dose that is at least 80-90% of the dose at the center.

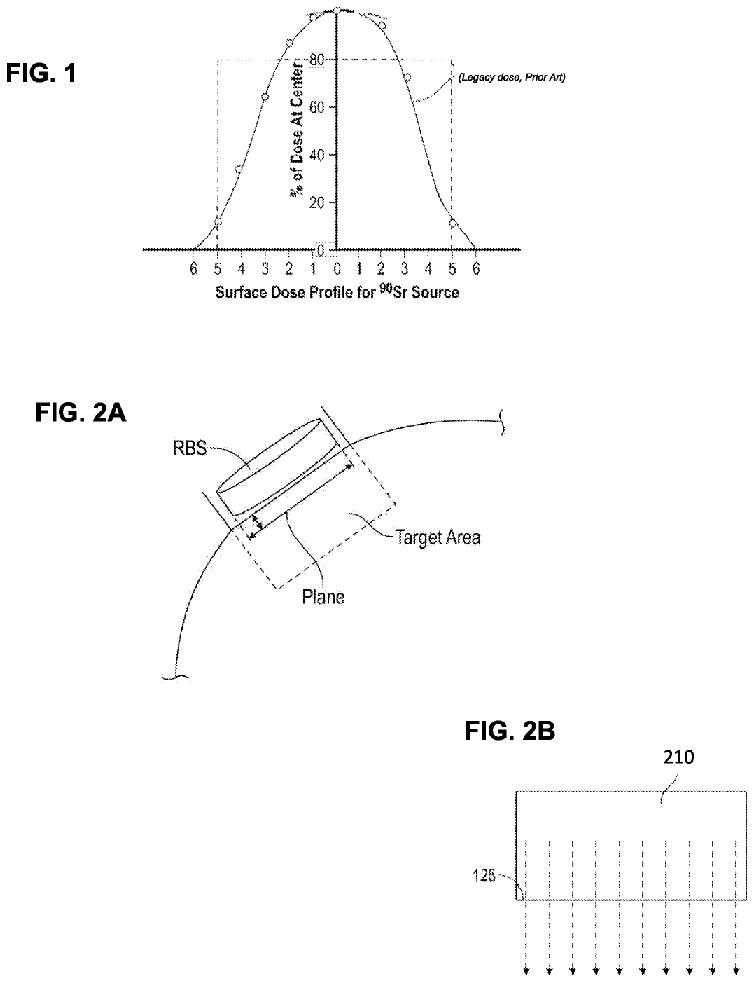

### DETAILED DESCRIPTION OF THE INVENTION

The present invention features brachytherapy systems for applying radiation to a target area. While the present invention describes applications of the systems and devices for treating glaucoma drainage bleb tissues or drainage holes, e.g., to help avoid scar formation or wound reversion, to inhibit or reduce fibrogenesis and/or inflammation in blebs or holes, etc., the present invention is not limited to the applications disclosed herein.

Briefly, the present invention provides radionuclide brachytherapy sources (RBSs) and RBS systems. The systems of the present invention provide a substantially uniform distribution of radiation from the system (e.g., the RBS system). As is described herein, the distribution of radiation that is emitted from the brachytherapy applicator may be determined by features of the RBS or RBS system. In certain embodiments, the RBS and/or source within is designed in a way, or comprises features, that help determine (e.g., optimize) the distribution of the dose of beta radiation to the target area, e.g., target plane of the treatment area. In certain embodiments, the radiation attenuation mask comprises features that help determine (e.g., optimizes) the distribution of the dose of beta radiation to the target area, e.g., target plane of the treatment area, such as radiation attenuation features. The radiation attenuation features (or radiation shaping features, flattening filters, etc.) may be integrated into the radiation attenuation mask, or they may be separate units from the radiation attenuation mask and/or RBS.

While the present invention describes applications of the systems and devices for treating glaucoma drainage bleb tissues or drainage holes, the present invention is not limited to the applications disclosed herein. For example, the systems and devices may feature applying beta radiation to ocular wounds, such as wounds due to the presence of a foreign body or trauma. Further, the systems and devices may feature applying beta radiation to non-ocular targets.

As used herein, the term “drainage device” refers to any or a combination of the general and specific approaches for draining aqueous humor, such as the therapeutics and devices described herein, including minimally invasive glaucoma surgery (MIGS) devices and surgery, that are employed to reduce Intraocular Pressure by means of a surgical intervention with a device.

Various glaucoma drainage procedures and devices, including trabeculectomy, drainage tubes, and devices used for Minimally Invasive Glaucoma Surgery (MIGS), are described herein or are well known to one of ordinary skill in the art. For the purposes of the invention, other surgical innovations and/or devices in addition to those described above may be included in the scope of the invention and described and labeled as MIGS. For example, techniques and devices that may alternatively be described as Moderately Invasive Glaucoma Surgery or Augmented Incisional Surgery is also included in the present invention.

### Isotopes and Radioactivity

The US Nuclear Regulatory Commission (USNRC) (https://www.nrc.gov/about-nrc/radiation/health-effects/measuring-radiation.html) defines radioactivity as “the amount of ionizing radiation released by a material. Whether it emits alpha or beta particles, gamma rays, x-rays, or neutrons, a quantity of radioactive material is expressed in terms of its radioactivity (or simply its activity), which represents how many atoms in the material decay in a given time period. The units of measure for radioactivity are the curie (Ci) and becquerel (Bq).” Activity in a radioactive-decay process is defined as the number of disintegrations per second, or the number of unstable atomic nuclei that decay per second in a given sample. Activity is expressed in the International System of Units by the becquerel (abbreviated Bq), which is exactly equal to one disintegration per second. Another unit that may be used is the Curie, wherein one curie is approximately the activity of 1 gram of radium and equals (exactly) 3.7×1010 becquerel. The specific activity of radionuclides is relevant when it comes to select them for production for therapeutic pharmaceuticals.

By the USNRC definition, absorbed dose is defined as the amount of radiation absorbed, e.g., the amount of energy that radioactive sources deposit in materials through which they pass or the concentration of energy deposited in tissue as a result of an exposure to ionizing radiation. The absorbed dose is equal to the radiation exposure (ions or Ci/kg) of the radiation beam multiplied by the ionization energy of the medium to be ionized. Typically, the units for absorbed dose are the radiation absorbed dose (rad) and gray (Gy). Gy is a unit of ionizing radiation dose defined as the absorption of one joule of radiation energy per kilogram of matter. The rad has generally been replaced by the Gy in SI derived units. 1 Gy is equivalent to 100 rad.

In some embodiments, the present invention features the use of Strontium 90 (Sr-90) in secular equilibrium with Yttrium 90 (Y-90). Strontium 90 (Sr-90) decays by beta radiation to Yttrium 90 (Y-90). The parent Sr-90 isotope has a half-life of 28.79 years. The daughter Y-90 isotope has a half-life of 64.0 hours. The two isotopes are in secular equilibrium with the decay rate of the combined source controlled by the Sr-90 parent but with the therapeutic beta radiations emanating from the daughter Y-90 with maximum energy of 2.28 MeV and an average energy of 934 keV.

In some embodiments, the present invention features the use of Yttrium-90 with a half-life of 64 hours. Y-90 decays to Zirconium 90 (Zr-90), a stable isotope, along three different routes via beta emission, wherein 99.985% of the time it decays with a maximum beta particle energy of 2.2801 MeV and a mean beta particle energy of 0.9337 MeV, or approximately or 1.5×10-13 joules. The other minor decay paths produce additional low energy gamma-rays, and electrons. Compared to the dominant path, the radiation doses from these paths are clinically negligible. In some embodiments, the present invention features the use of Phosphorus-132. [0017] In some embodiments, the present invention features the use of Ruthenium-106. In some embodiments, the present invention features the use of one or more radioactive isotopes of Cesium. In some embodiments, the present invention features the use of Cesium-131.

In some embodiments, the present invention features the use of one or more radioactive isotopes.

The Planning Target Volume (PTV) or Planning Treatment Volume (PTV) is a geometrical concept introduced for radiation treatment planning. The PTV is used to ensure that the prescribed dose is actually delivered to all parts of the target tissue. As an example, it would follow that the Target Volume could be defined as a disk of diameter 8 mm and depth of 0.2 mm, containing the tissue. In some embodiments, the target volume has a diameter of 8 mm and a depth of 0.5 mm. In some embodiments, the target volume has a diameter of 8 mm and a depth of 1 mm. In some embodiments, the target volume has a diameter of 8 mm and a depth of 1.5 mm. In some embodiments, the target volume has a diameter of 8 mm and a depth of 2 mm. In some embodiments, the target volume has a diameter of 10 mm and a depth of 0.2 mm. In some embodiments, the target volume has a diameter of 10 mm and a depth of 0.5 mm. In some embodiments, the target volume has a diameter of 10 mm and a depth of 1 mm. In some embodiments, the target volume has a diameter of 10 mm and a depth of 1.5 mm. In some embodiments, the target volume has a diameter of 10 mm and a depth of 2 mm.

For example, a prescription dose of brachytherapy of 10 Gray (1000 cGy) is 10 joules/kg absorbed dose throughout the Target Volume. Measurements have suggested a model Sr-90/Y-90 RBS with Activity of 1.48 GBq produces a surface dose rate of approximately 0.20 Gy per second. To deliver a dose of 10 Gy to the Target Volume would require an irradiation time of 50 seconds. The number of nuclei that decay during this 50 second treatment would be 1.48×109 Bq (disintegrations per second)×50 seconds=7.4×1010.

The unique dose prescription convention traditionally utilized in beta ophthalmic applicator brachytherapy specifies the maximum dose to the center point of the proximal surface. The dose to any point radial from the center dose, and/or at any depth in tissue, is a lower dose than the prescribed dose. For example, Soares et al has reported on the surface distribution of 90Sr+90Y ophthalmic applicators.

In a 1D prescription convention, the dose throughout the Planning Target Volume (PTV) is less than the prescribed dose at the proximal surface center area. For example, a PTV comprising a disc of equal diameter to a legacy Beta Applicator's active surface, and of shallow depth, (e.g., r=4 mm, z=0.5 mm), the Treated Volume (TV) is treated at a lower (unspecified) dose than the prescribed central surface dose.

Whereas conversely, for plaque brachytherapy the standard convention for dose prescriptions is the specification of the minimum dose to the distal apex of the lesion. For example, the Collaborative Ocular Melanoma Study (COMS) prescribes 85 Gy to the (distal) tumor apex. This prescribed dose is the minimum distal dose, whereas the proximal surface dose is larger.

Treated Volume (TV) is the volume of tissue enclosed within a specific isodose envelope enclosing the prescription dose. A non-limiting example of a 2D TV is the 80% isodose area. TV is not limited to 80% isodose areas and volumes. Beta radiation's significant dose attenuation with depth may require a different treatment dose at depth. For example, a 10 cGy dose near the proximal surface (e.g. depth z=0.2 mm) may provide for a therapeutic prescription dose of 6Gy at the depth z=0.6 mm.

Radiation is attenuated by distance and density (e.g. shielding). In addition, shorter exposure times provide for less received dose. Alpha particles are described as easily shielded. A thin piece of paper or several cm of air is usually sufficient to stop them. Beta particles are more penetrating than alpha particles. Beta shields are sometimes made of aluminum, brass, plastic, or other materials of low atomic number to reduce the production of bremsstrahlung radiation. Beta can also be shielded by higher atomic number materials. Gamma rays' linear attenuation coefficients are proportional to the absorber density.

Radiation dose can be modified by attenuation materials. The dose shape can be collimated by high density materials such as lead and other alloys. For example, a rapid method of production of irregular-shaped fields for use with patients receiving electron radiotherapy was described by Usher using a low melting point alloy (Plane J H, Usher C. A rapid method of production of irregular-shaped fields for use with patients receiving electron radiotherapy. Br J Radiol. 1990 November; 63(755):882-3.)

The differential dose rate across the diameter, or portion of the area thereof, can be modified with unequal mass-density paths (lengths).

One example is to vary the thickness of the same material over a portion of the area or diameter so as to alter the output dose over the area.

Therapeutic linear accelerators used in medicine are often fitted with a flattening filter. Faddegon B A, O'Brien P, Mason D L. The flatness of Siemens linear accelerator x-ray fields. Med Phys. 1999 February; 26(2):220-8 report on a flattening filter designed with Monte Carlo and was subsequently machined from brass and mounted on their Siemens linear accelerator MXE treatment unit x-ray fields. Their measurements demonstrate that the large field flattener extends the useful radius of the field.

Another method is to place material of various density over a portion of the area or diameter to alter the output dose over the area. For example, a portion of the field can be selectively modified by adding a denser (higher z) material.

### Biological Effects of Radiation

The biological effectiveness of radiation depends on the linear energy transfer (LET), total dose, fractionation rate, and radiosensitivity of the targeted cells or tissues. As radiation interacts with matter, it loses its energy through interactions with atoms in its direct path. In radiation therapy, LET is defined as the average amount of energy lost per defined distance in tissue, as in the energy deposited into a handful of cells. LET occurs at different rates in different tissues, and quantification of LET in cellular systems is an important component of determining correct dosage in radiology. Low LET radiations are X-rays, gamma rays and beta particles.

Radiation induced ionizations can act directly on the cellular molecules and cause damage, such as DNA damage. Radiation induced ionizations also can act indirectly, producing free radicals that are derived from the ionization or excitation of the water component of the cell. Exposure of cells to ionizing radiation induces high-energy radiolysis of H₂O water molecules into H+ and OH− radicals. These radicals are themselves chemically reactive, and in turn recombine to produce a series of highly reactive combinations such as superoxide (02) and peroxide (H₂02) that produce oxidative damage to molecules, such as DNA, within the cell. Ionizing radiation-induced DNA breaks represent one of the dominant mechanisms of action of beta brachytherapy.

Multiple pathways are involved in the cell after its exposure to ionizing radiation. In the cellular response to radiation, several sensors detect the induced DNA damage and trigger signal transduction pathways. The activation of several signal transduction pathways by ionizing radiation results in altered expression of a series of target genes.

The promoters or enhancers of these genes may contain binding sites for one or more transcription factors, and a specific transcription factor can influence the transcription of multiple genes. The transcription factors p53, nuclear factor κB (NF-κB), the specificity protein 1 (SP1)-related retinoblastoma control proteins (RCPs), two p53-dependent genes, GADD45 and CDKN1A, and genes associated with the NER pathway (e.g., XPC) are typically upregulated by ionizing radiation exposure. Interestingly, NF-κB activation has been shown to strongly depend on charged particles' LET, with a maximal activation in the LET range of 90-300 keV/p m.

Importantly, the transcribed subset of target genes is critical for the decision between resuming normal function after cell-cycle arrest and DNA repair, entering senescence, or proceeding through apoptosis in cases of severe DNA damage.

Arrest of the cell cycle is an important part of DNA damage response, facilitating DNA repair and maintenance of genomic stability. Regulators of cell cycle arrest are activated by phosphorylation by ataxia telangiectasia mutated (ATM) and ATR. For example, p53 has a short half-life and is stabilized in response to a variety of cellular stresses after phosphorylation by ATM. After exposure to ionizing radiation, phosphorylation of the serine residues 15 and 20 on p53 by checkpoint kinase 2 (CHK2) reduces its binding to MDM2, which in its bound state targets p53 for degradation by the proteasome pathway. Thus, dissociation of p53 from MDM2 prolongs the half-life of p53. Other proteins, such as Pin 1, Parc, and p300, and p300/CBP-associated factor (PCAF) histone acetyltransferases regulate the transactivation activity of p53. For efficient repair, especially in non-dividing cells, cellular levels of deoxyribonucleotides are increased during the DNA damage repair by p53-dependent transcriptional induction of the ribonucleotide reductase RRM2B (p53R2). It is accepted that the severity of DNA damage is the critical factor in directing the signaling cascade toward reversible cell cycle arrest or apoptosis. As part of the signaling cascade, the abundance of p53 protein, specific posttranslational modifications, and its interaction with downstream effectors, such as GADD45a or p21, may be responsible for directing the cellular response at this decision point.

Other pathways besides DNA and p53 can be involved in the cellular response to exposure to ionizing radiation. For example, ionizing radiation can produce reactive oxygen species (ROS) in the cytoplasm.

Low-dose radiotherapy (LD-RT) is known to exert an anti-inflammatory effect. In vitro models have revealed anti-inflammatory effects of LD-RT in doses ranging from 0.1-1.0 Gy on immune cells such as macrophages and neutrophils. Studies have also shown that low-dose radiation therapy has an anti-inflammatory effect involving diminished CCL20 chemokine expression and granulocyte/endothelial cell adhesion. An in vitro study by Khaw et al. (1991, British Journal of Ophthalmology 75:580-583) of beta irradiation of fibroblasts in culture found that “radiation reduces the proliferation of human Tenon's capsule fibroblasts. The doses of radiation which inhibited cell proliferation more than 50% (at day 7 and 14) and yet did not cause a decrease in the cell population were 500, 750, and 1000 rads.” The fibroblasts enter a period of growth arrest but do not die.

The present invention features systems and devices for the application of beta radiation used in combination with surgical procedures and/or implants (e.g., MIGS implants) as described herein. The brachytherapy provided by the systems and devices herein helps to prevent or reduce bleb scarring or failure to maintain a functioning bleb. Without wishing to limit the present invention to any theory or mechanism, it is believed that the brachytherapy devices and systems herein may help to inhibit or reduce inflammation and/or fibrogenesis by downregulating cellular (e.g., fibroblast) activity without cell death.

The application of beta radiation provides a medicament-like treatment, similar to a drug, wherein the beta radiation, when consumed by the cells, causes biological changes in signaling and gene transcription, thereby affecting cellular activity and growth, e.g., cell cycle arrest.

The present invention provides compositions or products that are radioactive compositions (sources of beta radiation). The radioactive composition has a therapeutic effect via the generation of beta radiation by, for example, the mechanisms previously discussed. In generating the beta radiation, radioactive composition is consumed (e.g., the product is gradually used up), in that the radioisotope atoms of the beta radioisotope brachytherapy source decay into other nuclides.

### Targets of the Eye

As previously discussed, the present invention provides systems and devices, e.g., ophthalmic applicator systems, brachytherapy systems, etc., for applying beta radiation, e.g., to a treatment area or target of the eye. In some embodiments, the target is a site of the bleb in an eye being treated for glaucoma with a MIGS implant or MIGS procedure. In some embodiments, the target is a site of the bleb in an eye treated with a trabeculectomy. In some embodiments, the target is a site of the bleb in an eye treated with minimally invasive micro sclerostomy (MIMS). In some embodiments, the target is a site of the hole in an eye treated with MIMS. In some embodiments, the target is a site of the implant that is surgically inserted into the eye for the purpose of treating glaucoma. In some embodiments, the target is a site of the eye associated with pterygium.

In some embodiments, the target comprises an entire bleb. In some embodiments, the target comprises a portion of a bleb. In some embodiments, the target area surrounds an end of the MIGS implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel.

In some embodiments, the target area is the entire bleb, e.g., the perimeter of the bleb, the center of the bleb, and the portions of the bleb in between the perimeter and the center. In some embodiments, the target area is the perimeter of the bleb, e.g., a ring-shaped target area. In some embodiments, the target is the perimeter of the bleb and a portion of the bleb next to the perimeter, e.g., the target may be annulus-shaped. In some embodiments, the target is a portion of the bleb in between the center and the perimeter. In some embodiments, the target is at least a portion of the center of the bleb. The present invention is not limited to the aforementioned descriptions of target areas. For example, in certain embodiments, the target is (or includes) tissue surrounding the rim of a drainage channel.

In certain embodiments, the target is scarring at the back end of the microtube. New generations of microtubes have been shown to be easier to insert and resulting in less follow-up than conventional drainage surgery called trabeculectomy. However, a disadvantage of the microtube procedure may be that the drainage comes out of one focal drainage point and it is easier for the tissue around one focal point to scar and encapsulate the outflow point with a small igloo-like dome of scar tissue. This occurs because the cells in the tissue around the tube end are stimulated by damaged blood and the contents of the aqueous fluid to divide and produce new collagen tissue. The fibroblast cells then divide multiple times (proliferate) and produce new tissue, then change into elongated cells which are full of contractile fibres that contract the tissue around the tip, forming the dome of scar tissue obstructing the flow of fluid. Without wishing to limit the present invention to any theory or mechanism, it is believed that beta radiation causes the fibroblast cells to go into a state of hibernation so they do not divide and cannot make collagen or contract the new collagen tissue.

In some embodiments, the target is a target other than that associated with MIGS/MIMS/trabeculectomy. In some embodiments, the ophthalmic target is other targets than those associated with glaucoma drainage surgery. In some embodiments the target is inflammation, autoimmune mediated pathologies, or vascular pathologies of the eye. In some embodiments, the target is associated with infections (for example, Herpes Simplex Keratitis or Tuberculous sclerokeratitis), Corneal ulcerations (for example, Moorens), Allergic disorders (for example, Vernal), benign or malignant Tumors (for example, Squamous Cell Carcinoma) or benign growths (for example, papillomas), Degenerations (for example, pterygium), Cicitarising disease (for example, pemphigoid), Inflammations (for example, meibomian gland), ocular manifestations of Stevens-Johnson syndrome, Drug-induced cicatrizing conjunctivitis, Ligneous conjunctivitis, Corneal Vascularization, Pterygia, Vernal Catarrh, Small papillomas of the eyelid, limbal carcinoma, ocular malignant melanoma, nevus pigmentosus of the conjunctiva, hemangioma, chalazion. In some embodiments, the target is in the orbit of the eye. The present invention includes other ophthalmic indications and is not limited to the aforementioned targets.

### Radionuclide Brachytherapy Source (RBS)

The RBS of the present invention is constructed in a manner that is consistent with the Federal Code of Regulations, but is not limited to the terms mentioned in the Code. For example, the RBS of the present invention may further comprise a substrate. Also, for example, in addition to being enclosed by the mentioned “gold, titanium, stainless steel, or platinum”, in some embodiments the radionuclide (isotope) of the present invention may be enclosed by a combination of one or more of “gold, titanium, stainless steel, or platinum”. In some embodiments, the radionuclide (isotope) of the present invention may be enclosed by one or more layers of an inert material comprising silver, gold, titanium, tantalum, titanium alloy, stainless steel, platinum, tin, zinc, nickel, copper, other metals, ceramics, glass, or a combination of these.

In some embodiments, a surface on the substrate is shaped in a manner to provide a controlled projection of radiation. The substrate may be constructed from a variety of materials. For example, in some embodiments the substrate is constructed from a material comprising, a silver, an aluminum, a stainless steel, tungsten, nickel, tin, zirconium, zinc, copper, a metallic material, a ceramic material, a ceramic matrix, the like, or a combination thereof. In some embodiments, the substrate functions to shield a portion of the radiation emitted from the isotope. The encapsulation may be constructed from a variety of materials, for example from one or more layers of an inert material comprising a steel, a silver, a gold, a titanium, a platinum, another biocompatible material, the like, or a combination thereof.

Without wishing to limit the present invention to any theory or mechanisms, it is believed that previous brachytherapy sources generally only treated the center part of the target or under-dose the peripheral area and/or overdose the center. The systems of the present invention generally provide a more uniform dose across the target area, e.g., across an area of a plane within the target area. In certain embodiments, the radionuclide brachytherapy source (RBS) may be designed and/or constructed to provide a more substantially uniform radiation dose across a plane within the target, e.g., as compared to previously constructed devices. In certain embodiments, a portion of the brachytherapy system (e.g., radiation attenuation mask, radiation attenuation shield, etc.) may be designed and/or constructed to provide a more substantially uniform radiation dose across the target, e.g., as compared to previously constructed devices. In certain embodiments, a portion of the brachytherapy system (e.g., radiation attenuation mask, radiation attenuation shield, etc.) and the RBS may be designed and/or constructed to provide a more substantially uniform radiation dose across the target, e.g., as compared to previously constructed devices. The present invention is not limited to the dosimetry described herein. For example, in some embodiments, the system (e.g., the radiation attenuation mask, the radiation attenuation shield, the radiation attenuation mask with an integrated radiation attenuation shield or flattening filter, etc.) is designed such that the dose received at the perimeter of the bleb is higher than that received at the center of the bleb.

Iterative computer simulations of output dosimetry may be used to determine an optimized design of device. Film dosimetry is a method of measuring radioactive delivery from a source and can be used to measure the dose across the target. It can also be used to calibrate or compare radioactive sources or to determine the homogeneity of the dose pattern.

The present invention provides a radionuclide brachytherapy source (RBS), e.g, a sealed radiological or radioactive source. In some embodiments, the RBS comprises a capsule (210) having a distal surface (212), a proximal surface (211) opposite the distal surface (212), and a side wall (215); and an active beta radioisotope material (220) (e.g., a substrate) encased in the capsule (e.g., encapsulation). The active beta radioisotope material (220) emits beta radiation through at least a portion of the distal surface (212) of the capsule (210).

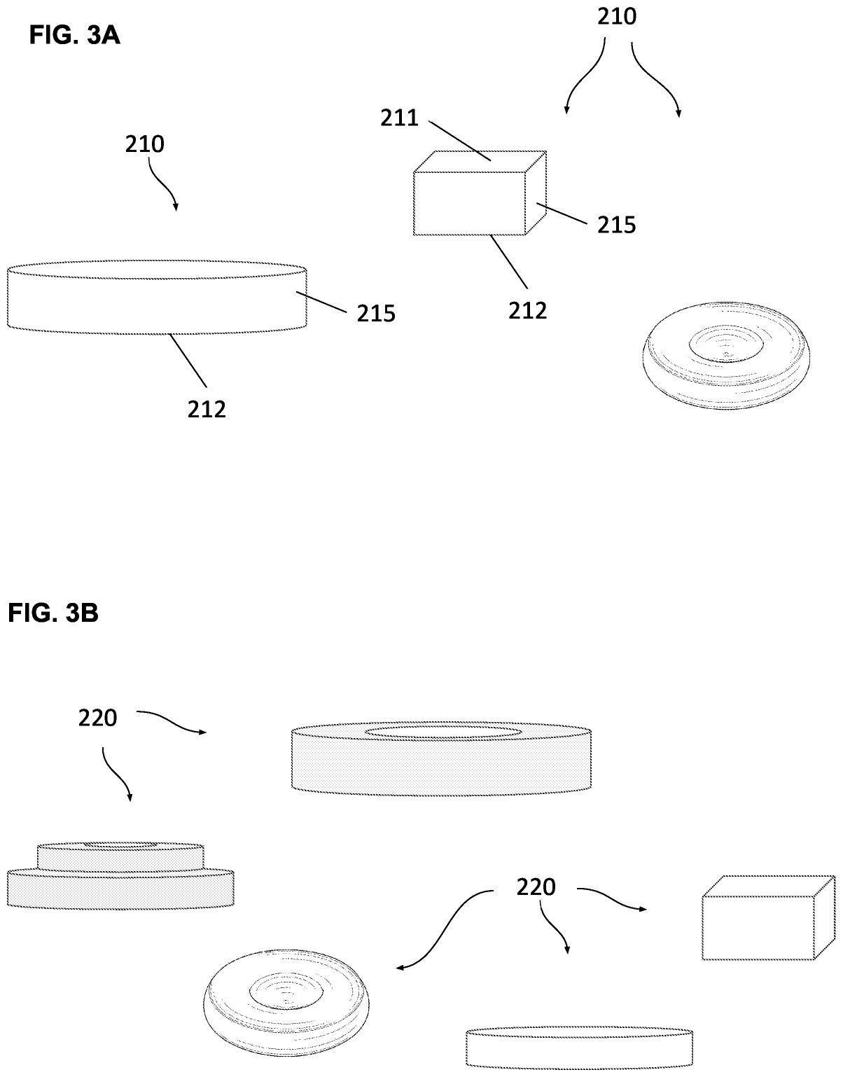

Referring to FIG. 3A, in some embodiments, the RBS (e.g., capsule) is cylindrical. In some embodiments, the RBS (e.g., capsule) is disc shaped, cuboidal, rounded, kidney-shaped, elliptical, etc. The present invention is not limited to those shapes, and any shape that achieves a desired dose profile is encompassed herein. The shape of the RBS may help provide a controlled projection of radiation (e.g., a therapeutic dose) onto the target. The shape of the RBS may help the radiation dose to fall off quickly at the periphery of the target (whatever the target is determined to be, e.g., the whole bleb, a portion of the bleb, etc.). This may help keep the radiation within a limited area/volume and may help prevent unwanted exposure of structures such as the lens to radiation.

Referring to FIG. 3B, in some embodiments, the active beta radioisotope material (220) is in a disc configuration. In some embodiments, the active beta radioisotope (220) is in an annulus configuration. The present invention is not limited to this configuration. In some embodiments, the radioisotope material (220) is in an alternative configuration, e.g., dome shaped, disc shaped, cuboidal, rounded, a round disc, kidney-shaped, elliptical, tiered, rectangular or cuboidal, a flattened dome, terraced, a truncated pyramid, a truncated cone, or trapezoidal, tier-shaped (e.g., wedding cake-shaped), or rounded tier-shaped, bean shaped, or any other appropriate shape. The present invention is not limited to those shapes, and any shape that achieves a desired dose profile is encompassed herein.

In some embodiments, the substrate (220) or active beta radioisotope material is any source of radiation, e.g., any source of beta radiation. In some embodiments, the substrate or active beta radioisotope material comprises Strontium-90 (Sr-90), Phosphorus-32 (P-32), Ruthenium 106 (Ru-106), Yttrium 90 (Y-90), Strontium-90 in secular equilibrium with Yttrium 90, an isotope of cesium (e.g., Cs-137), 1-125, or other radionuclides, or a combination thereof. The present invention also includes sources wherein both beta and gamma are emitted. The present invention also includes sources that provide gamma radiation but behave like beta, e.g., low energy or soft x-rays that behave similarly to beta (e.g, see, for example, Lee et al., 2008, Med. Phys. 35 (11) 5151-5160). In some embodiments, the isotope is coated on the substrate, and both the substrate and isotope are further coated with the encapsulation. In some embodiments, the radioactive isotope is embedded in the substrate. In some embodiments, the radioactive isotope is part of the substrate matrix. In some embodiments, the encapsulation may be coated onto the isotope, and optionally, a portion of the substrate. In some embodiments, the encapsulation is coated around the entire substrate and the isotope. In some embodiments, the encapsulation encloses the isotope. In some embodiments, the encapsulation encloses the entire substrate and the isotope. In some embodiments, the radioactive isotope is an independent piece and is sandwiched between the encapsulation and the substrate.

In some embodiments, the primary radionuclide in the source is Sr-90, which decays by beta radiation to Y-90. In some embodiments, the two isotopes are in secular equilibrium with the Sr-90 parent controlling the decay rate and the daughter Y-90 emanating the therapeutic beta radiations. In some embodiments, the decay produces an average energy of 934 keV and a maximum energy of 2.28 Mev. In some embodiments, the Sr-90 decays to Y-90 via beta emission 100% of the time with a maximum beta particle energy of 0.546 MeV and a mean beta particle energy of 0.1958 MeV. In some embodiments, the Y-90 decays to Zr-90 (stable isotope) along three different routes via beta emission. In some embodiments, 99.985% of the time with a maximum beta particle energy of 2.2801 MeV and a mean beta particle energy of 0.9337 MeV. In some embodiments, 0.0115% of the time with a maximum beta particle energy of 0.5194 MeV and a mean beta particle energy of 0.1856 MeV. This pathway may produce additional low energy gamma-rays, and electrons, but these are considered clinically negligible, especially for an encapsulated source. In some embodiments, 1.4×10⁻⁶% of the time with a maximum beta particle energy of 0.0938 MeV and a mean beta particle energy of 0.0250 MeV. This pathway may produce additional low energy gamma-rays, and electrons, but these are considered clinically negligible, especially for an encapsulated source. In some embodiments, the desired nominal Dose of 1000 cGy delivered at a depth of 200 μm from the conjunctiva surface in 30 seconds yields a dose rate of 33.3 cGy/s. In some embodiments, the allowed variation to achieve the desired dose is a treatment time of not less than 25 seconds (not greater than 40.0 cGy/s) and not more than 55 seconds (not less than 18.2 cGy/s)

In some embodiments, the dose rate will not vary by more than ±5% over the plane created at 400 μm water equivalent depth from the conjunctiva surface and a diameter 80% of the active material outer diameter. In some embodiments, the dose rate will be greater than 20 cGy/s through the volume created from the conjunctival surface to a depth of 600 μm water equivalent depth over a diameter at least 90% of the active material outer diameter. In some embodiments, the allowed variation of dose rate throughout the 200 μm to 600 μm depths of the PTV is between 33.3 cGy/s and 20 cGy/s. Practically this is a peak dose rate of 33.3 cGy at 200 μm, and a minimum dose not less than 20 cGy/s at 600 μm.

In some embodiments, the capsule is constructed from a material comprising stainless steel, gold, platinum, titanium, tantalum, titanium alloy, silver, tin, zinc, copper, nickel, aluminum, a ceramic, glass, a metal alloy, zirconium, or a combination thereof.

In some embodiments, the RBS (e.g., capsule (210)) has a diameter of 10.8 mm. In some embodiments, the RBS (e.g., capsule (210)) has a diameter from 4 to 20 mm. In some embodiments, the RBS (e.g., capsule (210)) has a diameter from 5 to 15 mm. In some embodiments, the RBS (e.g., capsule (210)) has a diameter from 10 to 20 mm. In some embodiments, the RBS (e.g., capsule (210)) has a diameter from 10 to 15 m. In some embodiments, the RBS (e.g., the capsule (210)) has a diameter from 5 to 7 mm (e.g., 5 mm, 6 mm, 7 mm). In some embodiments, the RBS (e.g., the capsule) has a diameter from 7 to 10 mm (e.g., 7 mm, 7.5 mm, 8 mm, 8.5 mm, 9 mm, 9.5 mm, 10 mm). In some embodiments, the RBS (e.g., the v) has a diameter from 9 to 12 mm (e.g., 9 mm, 9.5 mm, 10 mm, 10.5 mm, 11 mm, 11.5 mm, 12 mm). In some embodiments, the RBS (e.g., capsule (210)) has a diameter from 2 to 12 mm. In some embodiments, the RBS (e.g., the capsule (210)) has a diameter from 10 to 14 mm (e.g., 10 mm, 10.5 mm, 11 mm, 11.5 mm, 12 mm, 12.5 mm, 13 mm, 13.5 mm, 14 mm). In some embodiments, the RBS (e.g., the capsule (210)) has a diameter from 12 to 16 mm (e.g., 12 mm, 12.5 mm, 13 mm, 13.5 mm, 14 mm, 14.5 mm, 15 mm, 15.5 mm, 16 mm). In some embodiments, the RBS (e.g., the capsule) has a diameter from 14 to 18 mm (e.g., 14 mm, 14.5 mm, 15 mm, 15.5 mm, 16 mm, 16.5 mm, 17 mm, 17.5 mm, 18 mm). In some embodiments, the RBS (e.g., the capsule) has a diameter of 3 mm. In some embodiments, the RBS (e.g., the capsule) has a diameter of 4 mm. In some embodiments, the RBS has a diameter of 5 mm. In some embodiments, the RBS (e.g., the capsule) has a diameter of 5 mm. In some embodiments, the RBS (e.g., the capsule) has a diameter of 6 mm. In some embodiments, the RBS has a diameter of 7 mm. In some embodiments, the RBS (e.g., capsule (210)) has a diameter of 8 mm. In some embodiments, the RBS (e.g., capsule (210)) has a diameter of 9 mm. In some embodiments, the RBS (e.g., capsule (210)) has a diameter of 10 mm. In some embodiments, the RBS has a diameter of 11 mm. In some embodiments, the RBS (e.g., capsule (210)) has a diameter of 12 mm. In some embodiments, the RBS (e.g., capsule (210)) has a diameter of 13 mm. In some embodiments, the RBS (e.g., capsule (210)) has a diameter of 14 mm. In some embodiments, the RBS (e.g., capsule (210)) has a diameter of 15 mm. In some embodiments, the RBS has a diameter of 16 mm. In some embodiments, the RBS (e.g., capsule (210)) has a diameter of 17 mm. In some embodiments, the RBS (e.g., capsule (210)) has a diameter of 18 mm. In some embodiments, the RBS (e.g., capsule (210)) has a diameter of 19 mm. In some embodiments, the RBS (e.g., capsule (210)) has a diameter of 20 mm. In some embodiments, the RBS (e.g., capsule (210)) has a diameter more than 20 mm. In some embodiments, the RBS (e.g., capsule (210)) has a diameter from 10 to 100 mm. In some embodiments, the RBS (e.g., capsule (210)) has a diameter from 100 to 500 mm.

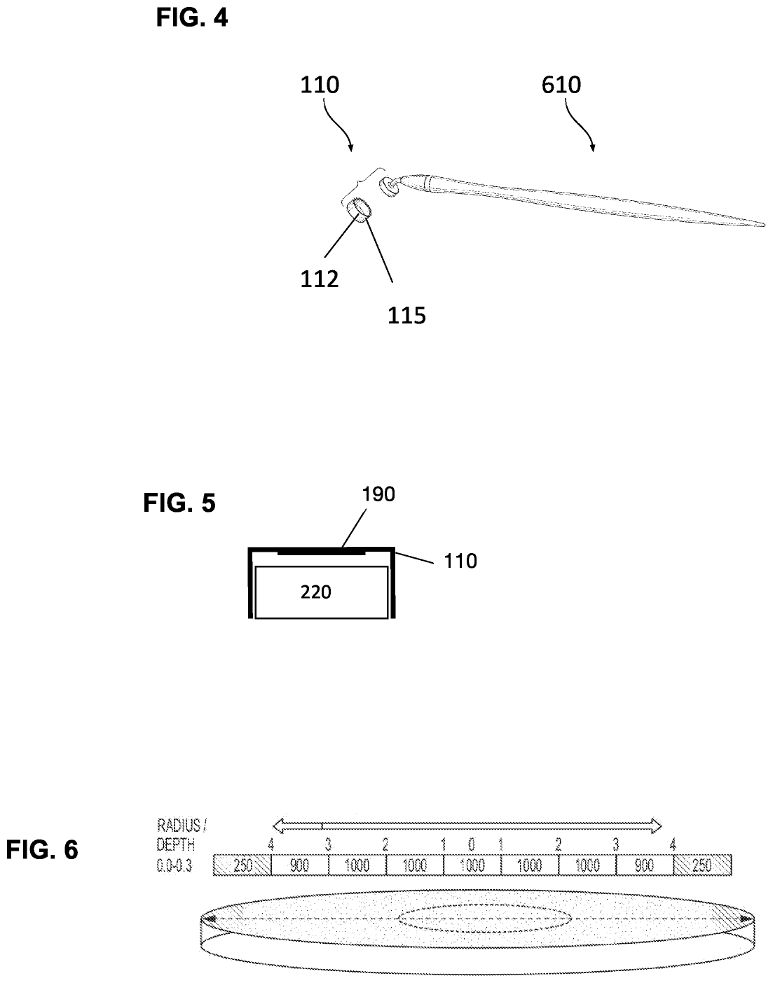

Referring to FIG. 4, the present invention also provides a brachytherapy applicator (610) and radiation attenuation mask (110) for housing a radionuclide brachytherapy source (RBS) (210), e.g., the radiation attenuation mask (110) can engage the applicator (610) (e.g., a distal end of the applicator) to house an RBS (210) therein. In certain embodiments, the applicator may feature the RBS fixedly attached to the applicator. For example, the applicator may be manufactured such that the RBS is integrated into the applicator prior to distribution or surgical use. In some embodiments, the applicator is manufactured to accept the RBS at a later time. For example, the applicator may be manufactured and distributed, and the RBS may be attached to or inserted into the applicator prior to its use in surgery, or engaged with the applicator via the radiation attenuation mask (110). The radiation attenuation mask may engage the brachytherapy applicator in any appropriate manner, e.g., snap mechanism, threaded mechanism, etc.

In some embodiments, the radiation attenuation mask (110) comprises an inner cavity formed by a side wall (115) and a bottom surface (112) sealed to a bottom edge (115c) of the side wall (115) around its perimeter, the inner cavity (140) is for accepting the RBS (210). In certain embodiments, the shape of the bottom surface may be a way of shaping the radiation. In some embodiments, the radiation attenuation mask is cylindrical, e.g, the side wall is cylindrical, the bottom surface is round. The radiation attenuation mask is not limited to a cylindrical configuration and may be shaped in any appropriate manner to accommodate an RBS.

The brachytherapy applicator (e.g., components of the brachytherapy applicator) may be constructed from any appropriate material, such as a biocompatible material or a combination of materials. Non-limiting examples of biocompatible materials include, but are not limited to, metals (for example, stainless steel, titanium, tantalum, titanium alloy, gold), ceramics and polymers. In certain embodiments, a component of the system or a portion thereof is constructed from a material comprising one or a combination of: stainless steel, titanium, copper, brass, tungsten, tungsten-copper, a metal alloy, or a polymer. In certain embodiments, a component of the system or a portion thereof is constructed from a material comprising a polymer. In certain embodiments, the polymer is one or a combination of: polycarbonate, PEEK, PEI, PET, PETG, ABS, Epoxy, Polyester, Polystyrene, polyurethane, PVDF, Polyimide, HIPS, or Styrene-butadienne rubber. In certain embodiments, a component of the system or a portion thereof is constructed from a material comprising stainless steel, titanium, tantalum, titanium alloy, gold, a ceramic, a polymer, or a combination thereof. In certain embodiments, a component of the system or a portion thereof is constructed from a material comprising a synthetic polymer material (e.g., plastic). In certain embodiments, a component of the system or a portion thereof is constructed from a material comprising a metal or metal alloy. The present invention is not limited to the particular materials described herein.

Generally, the applicator of the present invention may feature a handle and a distal portion at the end (e.g., distal end) of the handle for engaging and/or holding the radionuclide brachytherapy source (RBS) (e.g., radioisotope) and/or the radiation attenuation mask. The distal portion may be integrated into the distal end of the handle. In certain embodiments, the distal portion is removably attachable to the distal end of the handle.

The size of the radiation attenuation mask may be determined as appropriate for the RBS to be used in combination with the radiation attenuation mask. In some embodiments, the side wall or the inner cavity has a diameter from 7 to 14 mm. In some embodiments, the side wall or the inner cavity has a diameter of 12 mm or 13 mm. In some embodiments, the side wall has a height from 4 to 12 mm as measured from its bottom edge to its top edge. In some embodiments, the side wall has a height of 8.2 mm as measured from its bottom edge to its top edge. In some embodiments, the bottom surface of the radiation attenuation mask is 12 mm in diameter. In some embodiments, the bottom surface of the radiation attenuation mask is from 8 to 10 mm in diameter. In some embodiments, the bottom surface of the radiation attenuation mask is from 10 to 12 mm in diameter. In some embodiments, the bottom surface of the radiation attenuation mask is from 7 to 14 mm in diameter.

In certain embodiments, the radiation attenuation mask (110) is reusable. In certain embodiments, the radiation attenuation mask (110) is sterilizable.

Referring to FIG. 5, in certain embodiments, the radiation attenuation mask (110) comprises a radiation attenuation shield (190) for reducing radiation emitted from an RBS. The flattening filter (150) may be disposed on the bottom surface (112) in the inner cavity (140). The flattening filter (150) may be integrated into the bottom surface (112) in the inner cavity (140). In some embodiments, the flattening filter is a separate piece for placement on or in proximity to the bottom surface of the radiation attenuation mask (110). The flattening filter (150) reduces at least a portion of beta radiation emitted from an RBS thereby controlling an amount of beta radiation emitted from the bottom surface (112) of the radiation attenuation mask (110). In some embodiments, the flattening filter is annular, dome shaped, disc-shaped, a round disc, rectangular or cuboidal, a flattened dome, terraced, a truncated pyramid, a truncated cone, or trapezoidal, tier-shaped (e.g., wedding cake-shaped), or rounded tier-shaped, bean shaped, kidney shaped, or any other appropriate shape. The present invention is not limited to any particular shape or configuration of radiation attenuation shield.

### Target Plane

The system of the present invention delivers a dose of radiation to a target volume, e.g., a series of planes throughout a treatment volume (having a defined dimension such as a defined diameter). FIG. 2A shows an RBS or RBS system emitting radiation to a treatment volume (target area). Each plane is at a particular depth with respect to the RBS or RBS system. FIG. 2B shows the projection of radiation (125) from the RBS (210) or RBS system of the present invention.

In certain embodiments, the target plane or treatment volume has a diameter of about 2 mm. In certain embodiments, the target plane has a diameter of about 3 mm. In certain embodiments, the target plane or treatment volume has a diameter of about 4 mm. In certain embodiments, the target plane or treatment volume has a diameter of about 5 mm. In certain embodiments, the target plane or treatment volume has a diameter of about 6 mm. In certain embodiments, the target plane has a diameter of about 7 mm. In certain embodiments, the target plane or treatment volume has a diameter of about 8 mm. In certain embodiments, the target plane or treatment volume has a diameter of about 9 mm. In certain embodiments, the target plane or treatment volume has a diameter of about 10 mm. In certain embodiments, the target plane or treatment volume has a diameter of about 11 mm. In certain embodiments, the target plane or treatment volume has a diameter of about 12 mm. In certain embodiments, the target plane or treatment volume has a diameter from 10 to 14 mm. In certain embodiments, the target plane has a diameter from 6 to 10 mm. In certain embodiments, the target plane or treatment volume has a diameter from 5 to 12 mm. In certain embodiments, the target plane or treatment volume has a diameter from 6 to 12 mm. In certain embodiments, the target plane or treatment volume has a diameter from 8 to 10 mm. In certain embodiments, the target plane or treatment volume has a diameter from 8 to 12 mm. In certain embodiments, the target plane or treatment volume has a diameter from 6 to 8 mm. In certain embodiments, the target plane or treatment volume has a diameter from 7 to 10 mm. In certain embodiments, the target plane or treatment volume has a diameter from 8 to 11 mm. In certain embodiments, the target plane or treatment volume has a diameter from 9 to 11 mm. In certain embodiments, the target plane or treatment volume has a diameter from 9 to 12 mm. The present invention is not limited to the aforementioned dimensions of the target plane or treatment volume.

In certain embodiments, the target plane or treatment volume has a depth from 0 to 700 microns, e.g., as measured from the bottom surface of the RBS or of the RBS system (e.g., RBS and radiation attenuation mask). In certain embodiments, the target plane or treatment volume has a depth from 0 to 100 microns, e.g., as measured from the bottom surface of the RBS or of the RBS system (e.g., RBS and radiation attenuation mask). In certain embodiments, the target plane or treatment volume has a depth from 100 to 200 microns, as measured from the bottom surface of the RBS or of the RBS system (e.g., RBS and radiation attenuation mask). In certain embodiments, the target plane or treatment volume has a depth 200 to 400 microns, as measured from the bottom surface of the RBS or of the RBS system (e.g., RBS and radiation attenuation mask). In certain embodiments, the target plane or treatment volume has a depth from 200 to 600 microns, e.g., as measured from the bottom surface of the RBS or of the RBS system (e.g., RBS and radiation attenuation mask). In certain embodiments, the target plane or treatment volume has a depth from 400 to 600 microns, as measured from the bottom surface of the RBS or of the RBS system (e.g., RBS and radiation attenuation mask). In certain embodiments, the target plane or treatment volume has a depth from 0 to 1 mm. In certain embodiments, the target plane or treatment volume has a depth from 0.1 to 2 mm. In certain embodiments, the target plane or treatment volume has a depth from 0.2 to 1.6 mm. In certain embodiments, the target plane or treatment volume has a depth from 0.2 to 2. mm. Doses recited herein may refer to the doses at a particular depth from the surface of the device, for example at a depth of 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.9 mm, 1 mm, 1.1 mm, 1.2 mm, 1.3 mm, 1.4 mm, 1.5 mm, 1.6 mm 1.7 mm, 1.8 mm, 1.9 mm, 2.0 mm, 2.1 mm, 2.2 mm, 2.3 mm, 2.4 mm, 2.5 mm, etc.

### Dosing

The RBS and/or the RBS system delivers a particular radiation dose to the target, e.g., to a plane within the target (e.g., a plane of a certain size representing a portion of the treatment area (e.g., PTV)). In some embodiments, the system delivers a radiation dose of 1000 cGy (10Gy) to the target. In some embodiments, the system delivers a radiation dose of 900 cGy to the target. In some embodiments, the system delivers a radiation dose of 800 cGy to the target. In some embodiments, the system delivers a radiation dose of 750 cGy to the target. In some embodiments, the system delivers a radiation dose of 600 cGy to the target. In some embodiments, the system delivers a radiation dose of 500 cGy to the target. In some embodiments, the system delivers a radiation dose of 400 cGy to the target. In some embodiments, the system delivers a radiation dose of 300 cGy to the target. In some embodiments, the system delivers a radiation dose of 200 cGy to the target. In some embodiments, the system delivers a radiation dose of 100 cGy to the target. In some embodiments, the system delivers a radiation dose of 50 cGy to the target. In some embodiments, the system delivers a radiation dose of 1100 cGy to the target. In some embodiments, the system delivers a radiation dose of 1200 cGy to the target. In some embodiments, the system delivers a radiation dose of 1300 cGy to the target. In some embodiments, the system delivers a radiation dose of 1500 cGy to the target. In some embodiments, the system delivers a radiation dose from 600 cGy and 1500 cGy to the target. In some embodiments, the system delivers a radiation dose from 50 cGy to 100 cGy. In some embodiments, the system delivers a radiation dose from 100 cGy to 150 cGy. In some embodiments, the system delivers a radiation dose from 150 cGy to 200 cGy. In some embodiments, the system delivers a radiation dose from 200 cGy to 250 cGy. In some embodiments, the system delivers a radiation dose from 250 cGy to 300 cGy. In some embodiments, the system delivers a radiation dose from 300 cGy to 350 cGy. In some embodiments, the system delivers a radiation dose from 350 cGy to 400 cGy. In some embodiments, the system delivers a radiation dose from 400 cGy to 450 cGy. In some embodiments, the system delivers a radiation dose from 450 cGy to 500 cGy. In some embodiments, the system delivers a radiation dose from 500 cGy to 550 cGy. In some embodiments, the system delivers a radiation dose from 550 cGy to 600 cGy. In some embodiments, the system delivers a radiation dose from 600 cGy to 650 cGy. In some embodiments, the system delivers a radiation dose from 650 cGy to 700 cGy. In some embodiments, the system delivers a radiation dose from 700 cGy to 750 cGy. In some embodiments, the system delivers a radiation dose from 750 cGy to 800 cGy. In some embodiments, the system delivers a radiation dose from 800 cGy to 850 cGy. In some embodiments, the system delivers a radiation dose from 850 cGy to 900 cGy. In some embodiments, the system delivers a radiation dose from 900 cGy to 950 cGy. In some embodiments, the system delivers a radiation dose from 950 cGy to 1000 cGy. In some embodiments, the system delivers a radiation dose from 1000 cGy to 1050 cGy. In some embodiments, the system delivers a radiation dose from 1050 cGy to 1100 cGy. In some embodiments, the system delivers a radiation dose from 1100 cGy to 1150 cGy. In some embodiments, the system delivers a radiation dose from 1150 cGy to 1200 cGy. In some embodiments, the system delivers a radiation dose from 1200 cGy to 1250 cGy. In some embodiments, the system delivers a radiation dose from 1250 cGy to 1300 cGy. In some embodiments, the system delivers a radiation dose from 1300 cGy to 1350 cGy. In some embodiments, the system delivers a radiation dose from 1350 cGy to 1400 cGy. In some embodiments, the system delivers a radiation dose from 1400 cGy to 1450 cGy. In some embodiments, the system delivers a radiation dose from 1450 cGy to 1500 cGy. In some embodiments, the system delivers a radiation dose from 1500 cGy to 1550 cGy. In some embodiments, the system delivers a radiation dose from 1550 cGy to 1600 cGy. In some embodiments, the system delivers a radiation dose from 1600 cGy to 1800 cGy. In some embodiments, the system delivers a radiation dose from 1800 cGy to 2000 cGy. In some embodiments, the system delivers a radiation dose of 600, 650, 700, 750, 800, 850, 900, 950, 1000, 1050, 1100, 1150, 1200, 1250, 1300, 1350, 1400, 1450, or 1500 cGy to the target. In some embodiments, the system delivers a radiation dose of 1500 to 3200 cGy. In some embodiments, the system delivers a radiation dose of 3200 to 8000 cGy. In some embodiments, the system delivers a radiation dose of 8000 cGy to 10000 cGy. In some embodiments, the system delivers a radiation dose of greater than 10000 cGy.

As an example, in some embodiments, the dose rate at the surface of the RBS system (that is emitted to the treatment volume) is from 90 to 100 cGy/sec. In some embodiments, the dose rate at the surface of the RBS system (that is emitted to the treatment volume) is from 80 to 90 cGy/sec. In some embodiments, the dose rate at the surface of the RBS system (that is emitted to the treatment volume) is from 70 to 80 cGy/sec. In some embodiments, the dose rate at the surface of the RBS system (that is emitted to the treatment volume) is from 60 to 70 cGy/sec. In some embodiments, the dose rate at the surface of the RBS system (that is emitted to the treatment volume) is from 50 to 60 cGy/sec. In some embodiments, the dose rate at the surface of the RBS system (that is emitted to the treatment volume) is from 40 to 50 cGy/sec. In some embodiments, the dose rate at the surface of the RBS system (that is emitted to the treatment volume) is from 50 to 60 cGy/sec. In some embodiments, the dose rate at the surface of the RBS system (that is emitted to the treatment volume) is from 30 to 40 cGy/sec. In some embodiments, the dose rate at the surface of the RBS system (that is emitted to the treatment volume) is from 20 to 30 cGy/sec. In some embodiments, the dose rate at the surface of the RBS system (that is emitted to the treatment volume) is from 50 to 100 cGy/sec. In some embodiments, the dose rate at the surface of the RBS system (that is emitted to the treatment volume) is from 20 to 90 cGy/sec. The present invention is in no way limited to the aforementioned dose rates at the surface (within the diameter of the treatment volume). These values serve as examples only.

In some embodiments, the RBS and/or RBS system delivers the prescribed dose in a time from 10 seconds to 20 minutes. In some embodiments, the RBS and/or RBS system delivers the prescribed dose in a time from 20 seconds and 10 minutes. In some embodiments, the RBS and/or RBS system delivers the prescribed dose in a time from 20 seconds to 60 seconds. In some embodiments, the RBS and/or RBS system delivers the prescribed dose in a time from 30 seconds to 90 seconds. In some embodiments, the RBS and/or RBS system delivers the prescribed dose in a time from 60 seconds to 90 seconds. In some embodiments, the RBS and/or RBS system delivers the prescribed dose in a time from 90 seconds to 2 minutes. In some embodiments, the RBS and/or RBS system delivers the prescribed dose in a time from 2 minutes to 3 minutes.

In some embodiments, the RBS and/or RBS system delivers the prescribed dose in a time from 3 minutes to 4 minutes. In some embodiments, the RBS and/or RBS system delivers the prescribed dose in a time from 3 minutes to 5 minutes. In some embodiments, the RBS and/or RBS system delivers the prescribed dose in a time from 3 minutes to 6 minutes. In some embodiments, the RBS and/or RBS system delivers the prescribed dose in a time from 4 minutes to 5 minutes. In some embodiments, the RBS and/or RBS system delivers the prescribed dose in a time from 4 minutes to 6 minutes. In some embodiments, the RBS and/or RBS system delivers the prescribed dose in a time from 5 minutes to 6 minutes. In some embodiments, the RBS and/or RBS system delivers the prescribed dose in a time from 6 minutes to 7 minutes. In some embodiments, the RBS and/or RBS system delivers the prescribed dose in a time from 7 minutes to 8 minutes. In some embodiments, the RBS and/or RBS system delivers the prescribed dose in a time from 8 minutes to 9 minutes. In some embodiments, the RBS and/or RBS system delivers the prescribed dose in a time from 9 minutes to 10 minutes. In some embodiments, the RBS and/or RBS system delivers the prescribed dose in a time from 10 minutes to 12 minutes. In some embodiments, the RBS and/or RBS system delivers the prescribed dose in a time from 12 minutes to 15 minutes. In some embodiments, the RBS and/or RBS system delivers the prescribed dose in a time from 15 minutes to 20 minutes.

In some embodiments, the RBS and/or RBS system delivers the prescribed dose within 5 seconds. In some embodiments, the RBS and/or RBS system delivers the prescribed dose within 10 seconds. In some embodiments, the RBS and/or RBS system delivers the prescribed dose within 15 seconds. In some embodiments, the RBS and/or RBS system delivers the prescribed dose within 20 seconds. In some embodiments, the RBS and/or RBS system delivers the prescribed dose within 25 seconds. In some embodiments, the RBS and/or RBS system delivers the prescribed dose within 45 seconds. In some embodiments, the RBS and/or RBS system delivers the prescribed dose within 60 seconds. In some embodiments, the RBS and/or RBS system delivers the prescribed dose within 90 seconds. In some embodiments, the RBS and/or RBS system delivers the prescribed dose within 2 minutes. In some embodiments, the RBS and/or RBS system delivers the prescribed dose within 3 minutes. In some embodiments, the RBS and/or RBS system delivers the prescribed dose within 4 minutes. In some embodiments, the RBS and/or RBS system delivers the prescribed dose within 5 minutes. In some embodiments, the RBS and/or RBS system delivers the prescribed dose within 6 minutes. In some embodiments, the RBS and/or RBS system delivers the prescribed dose within 7 minutes. In some embodiments, the RBS and/or RBS system delivers the prescribed dose within 8 minutes. In some embodiments, the RBS and/or RBS system delivers the prescribed dose within 9 minutes. In some embodiments, the RBS and/or RBS system delivers the prescribed dose within 10 minutes. In some embodiments, the RBS and/or RBS system delivers the prescribed dose within 11 minutes. In some embodiments, the RBS and/or RBS system delivers the prescribed dose within 12 minutes. In some embodiments, the RBS and/or RBS system delivers the prescribed dose within 13 minutes. In some embodiments, the RBS and/or RBS system delivers the prescribed dose within 14 minutes. In some embodiments, the RBS and/or RBS system delivers the prescribed dose within 15 minutes. In some embodiments, the RBS and/or RBS system delivers the prescribed dose within 16 minutes. In some embodiments, the RBS and/or RBS system delivers the prescribed dose within 17 minutes. In some embodiments, the RBS and/or RBS system delivers the prescribed dose within 18 minutes. In some embodiments, the RBS and/or RBS system delivers the prescribed dose within 19 minutes. In some embodiments, the RBS and/or RBS system delivers the prescribed dose within 20 minutes. In some embodiments, the RBS and/or RBS system delivers the prescribed dose in a time frame greater than 20 minutes.

In some embodiments, a dose (e.g., a prescribed dose) may be delivered in a single application. In other embodiments, a dose (e.g., a prescribed dose) may be fractionated and applied in multiple applications. For example, in some embodiments, radiation (e.g., a prescribed dose) may be applied over the course of 2 applications. In some embodiments, radiation (e.g., a prescribed dose) may be applied over the course of 3 applications. In some embodiments, radiation (e.g., a prescribed dose) may be applied over the course of 4 applications. In some embodiments, radiation (e.g., a prescribed dose) may be applied over the course of 5 applications. In some embodiments, radiation (e.g., a prescribed dose) may be applied over the course of more than 5 applications. In some embodiments, radiation (e.g., a prescribed dose) may be applied over the course of 20 applications. In some embodiments, radiation (e.g., a prescribed dose) may be applied over the course of more than 20 applications.

Each application may deliver an equal sub-dose. In some embodiments, one or more of the sub-doses are different. For example, one or more of the sub-doses may be different so as to increase or decrease with each additional application.

According to one embodiment, a dose of radiation may be applied prior to the treatment procedure, e.g., surgery for implantation of a device, e.g., MIGS device, or other appropriate glaucoma procedure, e.g., MIMS. For example, in some embodiments, a dose of radiation may be applied one or more days prior to a surgery (e.g., insertion of a device, MIMS, etc.). In some embodiments, a dose of radiation may be applied within a 24-hour prior before a surgery (e.g., insertion of a device). In some embodiments, a dose of radiation may be applied just prior to a surgery (e.g., insertion of a device, MIMS, etc.), e.g., 1 hour before, 30 minutes before, 15 minutes before, 5 minutes before 1 minute before, etc. In some embodiments, a dose of radiation may be applied during a procedure, e.g., for implantation of a device. In some embodiments, a dose of radiation may be applied right after a surgery (e.g., implantation of a device (e.g., MIGS device), MIMS, etc.), e.g., within 1 minute, 2 minutes, 3 minutes, 5 minutes, 10 minutes, etc.). In some embodiments, a dose of radiation may be applied before an incision is made into the conjunctiva. In some embodiments, a dose of radiation may be applied after an incision is made into the conjunctiva. In other embodiments, a dose of radiation may be applied after a surgery (e.g., insertion of a device). In some embodiments, a dose of radiation may be applied within a 24-hour period after a surgery (e.g., insertion of a device). In some embodiments, a dose of radiation may be applied within one to two days after a surgery (e.g., insertion of a device). In some embodiments, a dose of radiation may be applied within 2 or more days after a surgery (e.g., insertion of a device). In some embodiments the dose may be applied any time after the glaucoma surgery. In some embodiments, the dose is applied months or years after the glaucoma surgery. For example, a dose may be given to patients that did not receive a dose during surgery but at a future date have scar or needling procedures to break up scar tissue.

### Dose Profile

FIG. 6 shows an example of a radiation dose profile and non-limiting examples of radiation dose profiles that are variations of that in FIG. 6A. The present invention provides a dose profile such that an area within 3-4 mm from the center gets a dose that is at least 80-90% of the dose at the center. Table 1 below provides examples of relative doses (relative to the center dose at that depth), based on FIG. 6 and a dose profile where the area within 3-4 mm from the center gets a dose that is at least 80-90% of the dose at the center.

TABLE 1

| | Distance from center of RBS (mm) | | | | | | | | |
|---|---|---|---|---|---|---|---|---|---|
| | 4 | 3 | 2 | 1 | 0 | 1 | 2 | 3 | 4 |
| % | 90 | 100 | 100 | 100 | 100 | 100 | 100 | 100 | 90 |
| dose | 80 | 90 | 90 | 90 | 100 | 90 | 90 | 90 | 80 |
| | 80+ | 80+ | 80+ | 80+ | 100 | 80+ | 80+ | 80+ | 80+ |
| | 90 | 90 | 100 | 110 | 100 | 110 | 100 | 90 | 90 |
| | 80- | 80-100 | 80- | 80- | 100 | 80- | 80- | 80-100 | 80- |
| | 100 | | 100 | 100 | | 100 | 100 | | 100 |
| | 90- | 90-100 | 90- | 90- | 100 | 90- | 90- | 90-100 | 90- |
| | 100 | | 100 | 100 | | 100 | 100 | | 100 |
| | 80- | 80-110 | 80- | 80- | 100 | 80- | 80- | 80-110 | 80- |
| | 110 | | 110 | 110 | | 110 | 110 | | 110 |
| | 80- | 80-120 | 80- | 80- | 100 | 80- | 80- | 80-120 | 80- |
| | 120 | | 120 | 120 | | 120 | 120 | | 120 |

FIG. 6B shows non-limiting examples of radiation doses at various points in the treatment volume The present invention is not limited to the doses described herein.

TABLE 2

| | Distance from center of RBS (mm) | | | | | | | | |
|---|---|---|---|---|---|---|---|---|---|
| | 4 | 3 | 2 | 1 | 0 | 1 | 2 | 3 | 4 |
| cGy | 250 | 900 | 1000 | 1000 | 1000 | 1000 | 1000 | 900 | 250 |
| | 800 | 900 | 900 | 900 | 1000 | 900 | 900 | 900 | 800 |
| | 720 | 810 | 810 | 950 | 900 | 950 | 810 | 810 | 720 |
| | 640 | 720 | 720 | 820 | 800 | 820 | 720 | 720 | 640 |
| | 580 | 650 | 650 | 750 | 725 | 750 | 650 | 650 | 580 |
| | 560 | 630 | 630 | 750 | 700 | 750 | 630 | 630 | 560 |
| | 520 | 580 | 580 | 650 | 650 | 650 | 580 | 580 | 520 |
| | 480 | 540 | 540 | 650 | 600 | 650 | 540 | 540 | 480 |
| | 650 | 725 | 750 | 725 | 725 | 725 | 700 | 725 | 650 |
| | 975 | 1000 | 1100 | 950 | 725 | 950 | 1100 | 1000 | 975 |
| | 650 | 700 | 700 | 700 | 600 | 700 | 700 | 700 | 650 |
| | 675 | 725 | 725 | 725 | 625 | 725 | 725 | 725 | 675 |
| | 775 | 875 | 900 | 775 | 700 | 775 | 900 | 875 | 775 |
| | 700 | 800 | 850 | 800 | 650 | 800 | 850 | 800 | 700 |
| | 700 | 750 | 800 | 775 | 675 | 775 | 800 | 750 | 700 |
| | 650 | 700 | 750 | 700 | 700 | 700 | 750 | 700 | 650 |

### Kits

The present invention also features kits comprising one or more components of the brachytherapy systems of the present invention. For example, in some embodiments, the kit comprises a radiation attenuation mask. In some embodiments, the kit comprises a brachytherapy applicator, e.g., the applicator without the RBS. In certain embodiments, the kit may comprise the applicator, e.g., a handle, and a radiation attenuation mask for engaging the handle once the RBS is inside the radiation attenuation mask. In some embodiments, the kit comprises a beta radiation source (e.g., RBS) and a brachytherapy applicator. In some embodiments, the kit comprises a portion of the components of the brachytherapy applicator. In some embodiments, the kit further comprises a radiation attenuation shield. In certain embodiments, the radiation attenuation shield is integrated into the radiation attenuation mask.

In some embodiments, the kit comprises a brachytherapy applicator (e.g., the handle portion and the radiation attenuation mask) and an implant for trans-scleral insertion (e.g., an implant for trans-scleral insertion that forms a bleb in the subconjuctival space of the eye (or forms a bleb in the space between the conjunctive and Tenon's capsule). In some embodiments, the kit comprises a brachytherapy applicator (e.g., the handle portion and the radiation attenuation mask), a radionuclide brachytherapy source, and an implant for trans-scleral insertion (e.g., an implant for trans-scleral insertion that forms a bleb in the subconjuctival space of the eye (or forms a bleb in the space between the conjunctive and Tenon's capsule). For example, in certain embodiments, the handle and radiation attenuation mask (e.g., cap) are provided in a kit packaged with a MIGS drainage device.

In some embodiments, the kit is for single use. The kit may be provided in sterile packaging.

### Methods

The systems and devices of the present invention may be used for a variety of methods. Non-limiting examples of methods of use of the systems and devices herein include methods for applying beta radiation to a target of the eye, for example the site of a bleb formed by a MIGS implant or procedure. Other methods include methods of inhibiting or fibrogenesis or inhibiting or reducing inflammation in a bleb or hole associated with a MIGS implant or procedure, a trabeculectomy, a MIMS procedure, etc.

As an example, the systems and devices of the present invention provide for a method of treating glaucoma drainage procedure conjunctival blebs with a substantially uniform dose of beta therapy, e.g., a substantially uniform dose of beta therapy across a diameter of about 10 mm, 9 mm, 8 mm, 7 mm, 6 mm, 5 mm, etc.

Other methods include methods to maintain the function of a bleb, methods to enhance the function of a MIGS implant, e.g., by maintaining a functional bleb, methods to enhance the success of MIMS, methods for repairing a failed trabeculectomy, methods for repairing a failed MIMS, methods to reduce intraocular pressure (IOP), methods to maintain a healthy IOP, methods for treating glaucoma, etc.

The methods herein comprise applying beta radiation to a target area of the eye. In some embodiments, the target area is a site of the bleb or an expected site of the bleb. (Note that the target is not limited to a bleb or a portion of a bleb.) In some embodiments, the target area surrounds the end of an implant. In some embodiments, the target is from 2 to 5 mm in diameter. In some embodiments, the target is from 5 to 12 mm in diameter. In some embodiments, the target is from 0.3 mm to 0.5 mm in thickness. In some embodiments, the target is from 0.01 mm to 0.7 mm in thickness. In some embodiments, the target is from 0.1 mm to 0.6 mm in thickness. The present invention is not limited to the aforementioned dimensions of the target.

In some embodiments, the method comprises applying the beta radiation prior to a particular surgical procedure, e.g., prior to insertion of a MIGS implant, prior to incision of the conjunctive, prior to creation of a hole associated with MIMS, etc. In some embodiments, the method comprises applying the beta radiation after a particular surgical procedure.

The present invention features methods for inhibiting or reducing fibrogenesis and inflammation in a bleb of an eye being treated for glaucoma. The present invention also provides methods for maintaining a functioning drainage bleb in the eye of a patient being treated for glaucoma. The present invention also features a method of treating glaucoma. The present invention also features a method of reducing intraocular pressure (IOP) in an eye. The present invention also features a method of reducing inflammation in an eye having a foreign body therein (e.g., the foreign body may be a Minimally Invasive Glaucoma Surgery (MIGS) implant inserted between an anterior chamber of the eye and a subconjunctival space of the eye or between the anterior chamber of the eye and a space between the conjunctiva and Tenon's capsule), wherein the implant causes formation of a bleb for draining aqueous humor.

The methods feature applying a therapeutic amount of beta radiation from a radioisotope to a target area of the eye using an applicator system, as described herein.

The beta radiation may be effective for reducing an Intraocular Pressure (IOP) of the eye, the beta radiation may be effective for treating glaucoma, the beta radiation may cause cell cycle arrest in fibroblasts on the Tenon's capsule to inhibit or reduce the fibrotic process and inflammation that leads to bleb failure, the beta radiation may reduce or inhibit a fibrotic process and inflammation that causes bleb failure, the beta radiation may help to effectively maintain the drainage function of the bleb.

In some embodiments, the method comprises implanting a Minimally Invasive Glaucoma Surgery (MIGS) implant within the eye, wherein the implant causes formation of a bleb (e.g., in the subconjunctival space of the eye, in a space between the conjunctiva and Tenon's capsule); the bleb functions to drain aqueous humor. In certain embodiments, the implant is inserted trans-sclerally, between an anterior chamber of the eye and a subconjunctival space of the eye, between the anterior chamber of the eye and a space between the conjunctiva and Tenon's capsule, etc.

In some embodiments, the methods herein comprise introducing a drug to a site, e.g., a site of the MIGS implant, a site of the bleb, a different part of the eye.

As previously discussed, ionizing radiation has effects on cells that can lead to cell cycle arrest. In some embodiments, the beta radiation of the present invention causes cell cycle arrest in fibroblasts on or associated with the Tenon's capsule or conjunctiva so as to inhibit or reduce the fibrotic process and inflammation that leads to bleb failure.

As previously discussed, the beta radiation may be applied via a radionuclide brachytherapy source (RBS). The RBS may be applied to the target via an applicator. As previously discussed, in some embodiments, the RBS provides a dose of about 750 cGy to the target. In some embodiments, the RBS provides a dose from 500 to 1000 cGy to the target.

The present invention also features methods for preparing an applicator for emitting beta radiation. In some embodiments, the method comprises inserting a radionuclide brachytherapy source (RBS) into an applicator, e.g., an appropriate place or cavity in the applicator. In some embodiments, the method comprises attaching the RBS to an applicator.

In some embodiments, the systems and devices of the present invention may be used for methods associated with needling procedures, e.g., procedures to the bleb to free or remove scar tissue and/or cystic structures in and/or around the bleb and/or surgery site that may later arise from wound healing or scarring or inflammatory responses to the glaucoma surgery. Needling procedures may affect surgical site morphology, restore the function of the surgery and/or lower the IOP.

Without wishing to limit the present invention to any theory or mechanism, it is believed that treating scar tissue formation on a bleb formed by a trabeculectomy procedure is different than treating a newly-created (and scar tissue-free) bleb at the time of the trabeculectomy. In some embodiments, the methods herein comprise applying beta therapy concomitant with a needling procedure to a bleb formed by a trabeculectomy procedure. In some embodiments, the methods herein comprise applying beta therapy to a trabeculectomy bleb that has formed scar tissue. In some embodiments, the methods herein comprise applying beta therapy to a bleb in the eye of a trabeculectomy patient where the intraocular pressure (IOP) has increased. In some embodiments, the methods herein comprise applying beta therapy to a bleb where the trabeculectomy is failing or has failed. In some embodiments, the methods herein comprise applying beta therapy to a bleb in a second trabeculectomy, where the first trabeculectomy has failed.

In some embodiments, the methods herein comprise applying beta therapy to a bleb that is failing or has failed. In some embodiments, the methods herein comprise applying beta therapy to a MIGS device bleb that is failing or has failed. In some embodiments, the methods herein comprise applying beta therapy to a MIGS device bleb that has formed scar tissue. In some embodiments, the methods herein comprise applying beta therapy to a bleb in the eye of a MIGS device patient where the intraocular pressure (IOP) has increased.

In some embodiments, the methods herein comprise applying another drug in addition to beta radiation to the eye, e.g., to the target, to an area near the target, etc. As a non-limiting example, the methods may further comprise administering pharmaceutical eyedrops or an anti-metabolite (e.g., a liquid anti-metabolite). In various embodiments, the drug may be administered before, during, or after the surgical implantation procedure. In some embodiments, the methods herein comprise applying another antimetabolite (e.g., mitomycin-c or 5-fluorouracil) in addition to beta radiation. In some embodiments, the methods comprise administering pharmaceutical eye drops or a liquid anti-metabolite or other liquid drug. In some embodiments, the drug is administered before, during, and/or after a surgical procedure.

The systems and devices (and methods) of the present invention may also be applied to wound healing, e.g., wounds in the eye due to foreign body insertion, trauma, ocular surface wounds, etc. One model of wound healing divides the process into hemostasis, inflammation, proliferation, and remodeling. The first phase of hemostasis begins immediately after wounding, with vascular constriction and fibrin clot formation. The clot and surrounding wound tissue release pro-inflammatory cytokines and growth factors such as transforming growth factor (TGF)-β, platelet-derived growth factor (PDGF), fibroblast growth factor (FGF), and epidermal growth factor (EGF). Once bleeding is controlled, inflammatory cells migrate into the wound and promote the inflammatory phase, which is characterized by the sequential infiltration of neutrophils, macrophages, and lymphocytes. In the early wound, macrophages release cytokines that promote the inflammatory response by recruiting and activating additional leukocytes. As macrophages clear these apoptotic cells, they undergo a phenotypic transition to a reparative state that stimulates keratinocytes, fibroblasts, and angiogenesis to promote tissue regeneration. T-lymphocytes migrate into wounds following the inflammatory cells and macrophages, and peak during the late-proliferative/early-remodeling phase. T-cells regulate many aspects of wound healing, including maintaining tissue integrity, defending against pathogens, and regulating inflammation. The proliferative phase generally follows and overlaps with the inflammatory phase, and is characterized by epithelial proliferation and migration over the provisional matrix within the wound (re-epithelialization). In the reparative dermis, fibroblasts and endothelial cells are the most prominent cell types present and support capillary growth, collagen formation, and the formation of granulation tissue at the site of injury. Within the wound bed, fibroblasts produce collagen as well as glycosaminoglycans and proteoglycans, which are major components of the extracellular matrix (ECM). Following robust proliferation and ECM synthesis, wound healing enters the final remodeling phase, which can last for years.

The radiation attenuation masks of the present invention reduce to acceptable medical practice the use of beta irradiation in trabeculectomy as a competitive first-choice therapy. This may be achieved both by: (1) the beta radiation source output is optimized to the Planning Treatment Volume(s) specific to the trabeculectomy surgical wound and bleb, and (2) minimizing stray dose to the lens, and thus empowering decreases in the side effects of induced cataract that otherwise limits selection of this treatment modality.

Of note, by convention dose variation is described as that across the diameter assuming a central point maximum dose. However, in practice it has been demonstrated that the maximum dose may be off center. Thus, the description of dose across the diameter may also include the variation of dose over the area.

As previously discussed, the present invention provides therapeutic doses of beta radiation, e.g., optimized doses of beta radiation, modified therapeutic doses of beta radiation, etc. The systems and devices of the present invention provide a relatively uniform dose across at least a portion of the target area, e.g., as described herein.

For example, the present invention also features a radionuclide brachytherapy source (RBS) system that emits beta radiation for use in a method of treating glaucoma. In some embodiments, the method comprises performing a glaucoma drainage surgery on an eye of a patient to form a bleb in a subconjunctival space or between the conjunctiva and Tenon's capsule, and to allow aqueous humor to drain into the bleb; and applying a modified therapeutic dose of beta radiation from the RBS system to a target area associated with the bleb, a drainage channel, a drainage implant, or a combination thereof.

The present invention also features a radionuclide brachytherapy source (RBS) system that emits beta radiation for use in a method of treating glaucoma (e.g., for helping to lower IOP). In some embodiments, the method comprises performing a glaucoma drainage surgery on an eye of a patient to form a bleb in a subconjunctival space or between the conjunctiva and Tenon's capsule, and to allow aqueous humor to drain into the bleb; and applying a modified therapeutic dose of beta radiation from the RBS system to a target area associated with the bleb, a drainage channel, a drainage implant, or a combination thereof.

The present invention also features a radionuclide brachytherapy source (RBS) system that emits beta radiation for use in a method of treating glaucoma (e.g., for helping to lower IOP). In some embodiments, the method comprises performing a glaucoma drainage surgery on an eye of a patient to form a bleb in a subconjunctival space or between the conjunctiva and Tenon's capsule, and to allow aqueous humor to drain into the bleb; and applying a modified therapeutic dose of beta radiation from the RBS system to a target area associated with the bleb, a drainage channel, a drainage implant, or a combination thereof.

The present invention also features a method of reducing intraocular pressure (IOP) in an eye being treated or having been treated with glaucoma drainage surgery to form a bleb in a subconjunctival space or between the conjunctiva and Tenon's capsule and to allow aqueous humor to drain into the drainage bleb. In some embodiments, the method comprises applying a modified therapeutic amount of beta radiation from a radionuclide brachytherapy (RBS) system to a target area associated with the bleb, a drainage channel, a drainage implant, or a combination thereof.

The present invention also features a method of treating glaucoma (e.g., to help effectively lower IOP, etc.), wherein the method comprises performing a glaucoma drainage surgery in an eye (e.g., MIGS, MIMS, trabeculectomy) to form a bleb in a subconjunctival space or between the conjunctiva and Tenon's capsule and to allow aqueous humor to drain into the drainage bleb; and applying a modified therapeutic amount of beta radiation from a radionuclide brachytherapy (RBS) system to a target area associated with the bleb, a drainage channel, a drainage implant, or a combination thereof.

In some embodiments, the methods herein are effective for lowering intraocular pressure (IOP). In some embodiments, the therapeutic amount of beta radiation helps maintain a functioning drainage bleb. In some embodiments, the therapeutic amount of beta radiation helps reduce conjunctival inflammation.

The present invention features methods for inhibiting or reducing fibrogenesis and inflammation in a bleb of an eye being treated for glaucoma. The present invention also provides methods for maintaining a functioning drainage bleb in the eye of a patient being treated for glaucoma. The present invention also features a method of treating glaucoma. The present invention also features a method of reducing intraocular pressure (IOP) in an eye. The present invention also features a method of reducing inflammation in an eye having a foreign body therein (e.g., the foreign body may be a Minimally Invasive Glaucoma Surgery (MIGS) implant inserted between an anterior chamber of the eye and a subconjunctival space of the eye or between the anterior chamber of the eye and a space between the conjunctiva and Tenon's capsule), wherein the implant causes formation of a bleb for draining aqueous humor.

The methods feature applying a therapeutic amount of beta radiation from a radioisotope to a target area of the eye using an applicator system, as described herein.

The beta radiation may be effective for reducing an Intraocular Pressure (IOP) of the eye, the beta radiation may be effective for treating glaucoma, the beta radiation may cause cell cycle arrest in fibroblasts on the Tenon's capsule to inhibit or reduce the fibrotic process and inflammation that leads to bleb failure, the beta radiation may reduce or inhibit a fibrotic process and inflammation that causes bleb failure, the beta radiation may help to effectively maintain the drainage function of the bleb.

In some embodiments, the method comprises implanting a Minimally Invasive Glaucoma Surgery (MIGS) implant within the eye, wherein the implant causes formation of a bleb (e.g., in the subconjunctival space of the eye, in a space between the conjunctiva and Tenon's capsule); the bleb functions to drain aqueous humor. In certain embodiments, the implant is inserted trans-sclerally, between an anterior chamber of the eye and a subconjunctival space of the eye, between the anterior chamber of the eye and a space between the conjunctiva and Tenon's capsule, etc.

In certain embodiments, the RBSs and systems herein are used to provide dose fractionation, wherein the RBS or system is rotated a certain number of times over the course of the application. This may help provide for a more even distribution of radiation.

In some embodiments, the applicator system is placed in contact with the eye at the target area and pressed upon, the distance from the outer surface of the distal end of the applicator system and the bottom surface of the bleb is substantially uniform across the target area.

In some embodiments, when the applicator system is placed in contact with the eye at the target area and pressed upon, at least 50% of the surface area of the outer surface of the distal end is in contact with the eye. In some embodiments, when the applicator system is placed in contact with the eye at the target area and pressed upon, at least 60% of the surface area of the outer surface of the distal end is in contact with the eye. In some embodiments, when the applicator system is placed in contact with the eye at the target area and pressed upon, at least 70% of the surface area of the outer surface of the distal end is in contact with the eye. In some embodiments, when the applicator system is placed in contact with the eye at the target area and pressed upon, at least 80% of the surface area of the outer surface of the distal end is in contact with the eye. In some embodiments, when the applicator system is placed in contact with the eye at the target area and pressed upon, at least 90% of the surface area of the outer surface of the distal end is in contact with the eye. In some embodiments, when the applicator system is placed in contact with the eye at the target area and pressed upon, at least 90% of the surface area of the outer surface of the distal end is in contact with the eye. In some embodiments, when the applicator system is placed in contact with the eye at the target area and pressed upon, at least 95% of the surface area of the outer surface of the distal end is in contact with the eye. In some embodiments, when the applicator system is placed in contact with the eye at the target area and pressed upon, at least 99% of the surface area of the outer surface of the distal end is in contact with the eye.

### Example 1: Surgical Procedure for Beta Radiation Application

The present invention provides an example of a procedure for the application of beta radiation to the eye. The present invention is in no way limited to the specific steps, methods, devices, systems, and compositions described herein.

### Preparation and Assembly

The device assembly procedure may be done behind a plexiglass beta shield (for example, the Large Dual Angle Beta Radiation Shield, Universal Medical Inc.). The medical technician or medical physicist or other user opens the Radioisotope Brachytherapy Source (RBS) storage container. The RBS is removed from its container using appropriate handling techniques (for example, long forceps). The RBS is placed on a clean field.

The Brachytherapy Applicator may be a single-use sterile-packed device. Its packaging may be checked by examining for damage or breach of the sterile barrier. If finding none, the Brachytherapy Applicator package is opened, and the applicator assembly placed on a sterile field.

The Brachytherapy Applicator comprises a handle and an RBS cap. Using aseptic technique and remote handling techniques, the RBS is loaded into the Brachytherapy Applicator, e.g., the RBS may be inserted into the cap and the handle is subsequently connected to the cap, securing the RBS. Care is taken to avoid contamination.

The radiation output may be confirmed consistent with standards of quality assurance in radiation therapy (for example see: Palmer, Antony L., Andrew Nisbet, and David Bradley. “Verification of high dose rate brachytherapy dose distributions with EBT3 Gafchromic film quality control techniques.” Physics in medicine and biology 58.3 (2013): 497). In one method of quality assurance, the applicator is applied to radiographic film in sterile overwrap for a specified dwell time (for example Gafchromic@film, Ashland Inc.). The overwrap is removed. The medical physicist checks the area of application for evidence of film exposure.

The device may be placed into a sterile plexiglass beta transport box (for example the IBI Beta-Gard Acrylic Storage Container—Large, Universal Medical Inc.) and the box placed on the operative Mayo stand.

Previously the decayed activity of the RBS has been calculated to determine the contemporary dose per unit time (for example, cGy/second). The decay calculation methodology is known to those skilled in medical physics and is also described in the NRC Information Notice 96-66: United States Nuclear Regulatory Commission, Office of Nuclear Material Safety and Safeguards, Washington D.C. 20555, Dec. 13, 1996. The dwell time for the total prescribed dose is then calculated. As an example, the prescription dose is 1,000 cGy to a center point of 0.19 mm depth from the conjunctival surface. As an example, the decayed activity of the RBS is 30 cGy/second at a water equivalent depth of 0.19 mm. In this example, the dwell time is calculated to be about 33 seconds, providing a 990 cGy dose.

### Surgical Application

The beta therapy may be applied following completion of a glaucoma surgery. (Note the present invention is not limited to applying beta radiation after glaucoma surgery.) The eye is rotated to a downward gaze position by the use of a probe placed against the sclera providing traction (for example the distal end of a Vera Hook placed against the eye). This allows better visual and surgical access to the superior conjunctiva.

The ophthalmic surgeon obtains the Brachytherapy Applicator device, e.g., from the transport box. The tip (e.g., distal end, active end) of the applicator is placed over the conjunctiva in a position just superior to the limbus. The diameter of the applicator encompasses the appropriate surface area of the target, e.g., bleb. The Brachytherapy Applicator is pressed to the surface of the eye. In some embodiments, the Brachytherapy Applicator is pressed to the surface of the eye such that all or substantially all of the edema fluid is pushed away. The Applicator is held in place for the specified dwell time. In some embodiments, the dwell time has been programmed into a count-down clock. Following the specified dwell time, the Brachytherapy Applicator is removed from the operative field.

At the conclusion of surgery, antibiotic ointment is applied to the eye and the eye patched.

In certain embodiments, following the surgery, the Brachytherapy Applicator is disassembled behind the acrylic beta shield. The Radioisotope Brachytherapy Source is returned to its storage container. The disposable portions of the device are discarded in a manner consistent with appropriate disposal of biological waste (for example “red bag” waste).

### Example 2: Application of Beta Radiation

The present invention provides an example of the application of beta radiation to the eye. The present invention is in no way limited to the specific steps, methods, devices, systems, and compositions described herein.

### Assembly and Disassembly

Using aseptic technique, insert the beta radiation source (which may be connected to an applicator) into the cavity of the radiation attenuation mask, taking care not to cross-contaminate the outer surface of the radiation attenuation mask with the beta radiation source and/or attached applicator.

Apply vertical force down until the radiation attenuation mask is seated fully, and an audible click is heard.

Verify the radiation attenuation mask is securely attached by visual inspection. Inspect that the source is fully seated in the radiation attenuation mask.

To dissemble the radiation attenuation mask and beta radiation source after a procedure is complete, using forceps, place the prongs on either side of the attached applicator handle on the radiation attenuation mask ledge perimeter.

Using the forceps to apply downward force on the radiation attenuation mask, pull the applicator with beta radiation source up until the radiation attenuation mask and beta radiation source separate. As soon as separation occurs stop pulling. The top of the source will hit the bottom of the forceps—ensure the force applied is not excessive.

Remove the beta radiation source from the radiation attenuation mask.

### Use

With the radiation attenuation mask affixed, the beta radiation source dose rate is decreased. The time required to deliver a specific dose (dwell time) will increase compared to the dwell time of the beta radiation source without the radiation attenuation mask affixed.

The medical physicist or responsible person verifies the relative dose distribution and absolute dose rate of the beta radiation source as modified by the radiation attenuation mask, according to established protocols.

The operator may find the dwell time to be on the order of 2.5×, 3×, 3.5×, etc. the previous dwell time for the beta radiation source alone. This safety-check time factor is not to be used for patient treatment planning.

In certain embodiments, the application of the beta radiation source with the radiation attenuation mask be fractionated. For example, in some embodiments, the application is fractionated into 4×¼ dwell time serial applications, each with an incremental 90-degree rotation. The operator may advance the rotation without lifting the applicator off the application site. Clinical experience has demonstrated that some original equipment manufacturer (OEM) supplied beta radiation sources wherein the maximum dose rate is not necessarily centered on the device axis. For such beta radiation sources, the off-axis dose rate maximum interaction with the radiation attenuation mask central attenuation feature may produce an off-axis area of maximum dose rate. The rotational application spreads the off-axis maximum dose-rate over the intermediary circumference, thus providing a more uniform dose delivery.

Following physician practice, internal institutional procedures and surgical instructions, place the radiation attenuation mask on the treatment application site for the predetermined length of time and in the predetermined manner (e.g., fractionation).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

As used herein, the term “about” may refer to plus or minus 10% of the referenced number, or a number within a range that would be considered equivalent to the recited value by one of ordinary skill in the art.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A radionuclide brachytherapy source (RBS) comprising a beta radioisotope component encased in a capsule wherein at least a portion of a bottom surface of the RBS is an active area, the active area being where beta radiation is emitted from, wherein an area within 3-4 mm from the center of the active area of the bottom surface of the RBS emits a dose that is at least 80% of the dose emitted at the center of the active area.

2. The RBS of claim 1, wherein the RBS is cylindrical.

3. The RBS of claim 1, wherein the beta radioisotope component is in a disc configuration.

4. The RBS of claim 1, wherein the beta radioisotope component is in an annulus configuration.

5. The RBS of claim 1, wherein the RBS has a diameter from 4 to 15 mm.

6. The RBS of claim 1, wherein the active area is from 6 to 12 mm in diameter.

7. The RBS of claim 1, wherein the beta radioisotope comprises Strontium-90 (Sr-90).

8. The RBS of claim 1, wherein the beta radioisotope comprises Yttrium 90 (Y-90).

9. The RBS claim 1, wherein the beta radioisotope comprises Strontium-90 in secular equilibrium with Yttrium 90.

10. The RBS of claim 1 further comprising a radiation attenuation mask for housing a radionuclide brachytherapy source (RBS), the radiation attenuation mask comprises a side wall, an open top end for accepting the RBS, and a bottom end sealed with the side wall along an entire bottom circumference of the side wall, forming an inner cavity.

11. The RBS of claim 10, wherein the radiation attenuation mask further comprises an attenuation component disposed in the inner cavity on the bottom surface, the dose flattening filter prevents a portion of beta radiation emitted from an active area of the RBS from passing through the bottom surface, thereby controlling an amount of beta radiation emitted from the bottom surface of the radiation attenuation mask.

12. The RBS of claim 10, wherein the radiation attenuation mask is cylindrical.

13. The RBS of claim 11, wherein the attenuation component is annulus shaped.

14. The RBS of claim 11, wherein the attenuation component is dome-shaped.

15. The RBS of claim 10, wherein the bottom surface of the radiation attenuation mask is from 7 to 14 mm in diameter.

16. The RBS of claim 1 having a dose profile wherein all points of a treatment volume within a 4 mm radius has a dose that is at least 80% of the dose at the center, the points of the treatment volume being at a depth relative to the RBS system.

17. The RBS of claim 16, wherein the depth is 0.2 to 1 mm.

18. The RBS of claim 16, wherein the depth is from 1 to 1.5 mm.

19. The RBS of claim 16, wherein the dose profile is therapeutic.

20. The RBS of claim 16, wherein the target is a target in an eye.
